(19) United States
(12) Patent Application Publication
(10) Pub. No.: US 2009/0148904 A1
(43) Pub. Date: Jun. 11, 2009

(54) PRODUCTION OF CYTOTOXIC ANTIBODY-TOXIN FUSION IN EUKARYOTIC ALGAE

(76) Inventor: Stephen P. Mayfield, Cardiff-by-the-Sea, CA (US)

Correspondence Address: DLA PIPER LLP (US), 4365 EXECUTIVE DRIVE, SUITE 1100, SAN DIEGO, CA, 92121-2133, US

(21) Appl. No.: 12/269,806

(22) Filed: Nov. 12, 2008

Related U.S. Application Data
(60) Provisional application No. 60/987,726, filed on Nov. 13, 2007.

(51) Int. Cl. C12P 21/04; C12N 15/11; C12N 5/04; C07K 16/18; C12N 5/06; C12N 1/13; C12N 15/00

(52) U.S. Cl. 435/69.6; 536/23.4; 435/419; 435/257.2; 435/320.1; 435/69.7; 435/317.1; 530/387.3; 435/375

(57) ABSTRACT

Methods and compositions are disclosed to engineer chloroplast comprising heterologous genes encoding target binding domain fused to a eukaryotic toxin and produced within a subcellular organelle, such as a chloroplast. The present disclosure demonstrates that when chloroplasts are used, toxins normally refractive to production in eukaryotic cells may be used to produce recombinant fusion proteins with binding domains that are soluble, properly folded and post-translationally modified, where the multifunctional activity of the fusion protein is intact. The binding domains may include those from antibodies, receptors, hormones, cytokines, chemokines, and interferons. The present disclosure also demonstrates the utility of plants, including green algae, for the production of complex multi-domain proteins as soluble bioactive therapeutic agents.

### CROSS REFERENCE TO RELATED APPLICATION(S)

This application claims the benefit of priority under 35 U.S.C. §119(e) of U.S. Ser. No. 60/987,726, filed Nov. 13, 2007, the entire content of which is incorporated herein by reference.

### GRANT INFORMATION

This invention was made with government support under Grant No. 1RO1 AI059614-01 A1 awarded by the National Institutes of Health. The government has certain rights in this invention.

### BACKGROUND OF THE INVENTION

1. Field of the Invention

The present invention relates generally to methods and compositions for expressing polypeptides in chloroplasts, and more specifically to antibody-toxin fusion constructs that encode therapeutic products that are expressed in chloroplasts.

2. Background Information

Protein based therapeutics, or biologics, are the fastest growing sector of drug development, mainly due to the efficacy and specificity of these molecules. The specificity of biologics comes from their complexity, and because biologics are only produced in living cells, the production of these molecules can be time consuming and expensive.

Previous monoclonal antibody-based therapies have been developed in which antibody binding to cell surface proteins results in activation of antibody-dependent cell-mediated cytotoxicity (ADCC) or antibody-toxin conjugates are used that are capable of directly killing targeted cells. In ADCC, for example, an immune response is activated where antibodies coat a target cell thereby marking them for attack by natural killer (NK) cells. Therapeutics based on ADCC have been shown to be effective for the treatment of several types of cancers. For fusions of antibodies and antibody fragments to chemical or protein toxins that involve direct killing targeted cells, such immunotoxins are usually constructed by chemically conjugating a cell toxic agent to an antibody directed against a cell surface protein known to internalize after antibody binding. Once internalized within the cell, the toxin is able to disrupt a vital cellular function such protein synthesis, leading to death of the target cell.

Although the utility of these types of molecules seems to have many applications, only one cancer drug using this strategy is presently on the market (Mylotarg®, Wyeth-Ayerst Laboratories). One reason for the failure of these hybrid molecules to be utilized more often may lie in the complex nature of their construction, with the antibody half of the molecule typically produced in mammalian cells and the toxin half of the molecule produced in bacteria or by chemical synthesis, followed by chemically linking of the two parts to one another in ratios of one or more toxin moieties per antibody. Chemically coupling a small molecule or protein toxin to an antibody suffers from several limitations. First, there are limited chemistries available for coupling of a small molecule or protein toxin to an antibody, and by the efficiencies of these chemical reactions. Second, the chemical coupling can be limited by the availability of suitable sites for attachment to the antibody, potentially resulting in the production of antibody-toxin fusions where the antibody portion of the molecule is rendered inactive. Once administered, the coupled toxin could dissociate prematurely from the antibody prior to internalization resulting in off-target cytotoxicity and reduced cell-killing at the target site by competition of the uncoupled antibody for cell surface binding sites with intact antibody-toxin conjugates.

An alternative approach for the production of antibody-toxin conjugates is the construction of genetic fusions, where an antibody coding region is genetically linked to the coding region of a protein toxin. Production of these types of fusion proteins is strictly limited to prokaryotic expression systems, as an active immunotoxin would kill any susceptible eukaryotic host. There are other limitations to this type of approach as well, because prokaryotic systems are typically unable to express full-length antibodies, and even the production of antibody fragments, such as scFvs and Fabs as fusions, fused to protein toxin domains is problematic as these domains are often insoluble in E. coli expression systems. This insolubility results in poor yields of active molecules and in time consuming and expensive protocols for solubilizing and re-folding of aggregated proteins from bacterial inclusion bodies.

Many protein-based eukaryotic toxins target the translational machinery of eukaryotic cells, specifically the 80S ribosome and cytoplasmic translational initiation and elongation factors. Protein toxins can be produced in prokaryotes because the translation machinery of bacteria is substantially different than that of eukaryotic cells in general. In a similar fashion, the translational apparatus of plant and algal plastids is fundamentally different from the translation machinery in other eukaryotic cytoplasm. Plastids contain prokaryotic-like 70S ribosome and associated translational factors that are very different from those present in the typical eukaryotic cytosol. Consequently, the chloroplast presents a unique environment for the production of eukaryotic toxins and for the production of antibody-toxin fusions, as plastids have evolved to contain a suite of molecular chaperones and redox factors capable of modulating complex protein folding and assembly, including formation of disulfide bonds.

By generating antibody-toxin fusion proteins as genetic fusions, instead of as chemical fusions, the production of these complex molecules can be greatly facilitated, making it possible to produce immunotoxin molecules with superior properties.

The expression of biologics in algae offers an attractive alternative to traditional mammalian-based expression systems, as the production of proteins in algae has inherently low costs of capitalization and production, and stable transgenic lines can be generated in a short period of time.

### SUMMARY OF THE INVENTION

The present invention discloses a method to generate therapeutic fusion proteins containing toxins, where these fused molecules are capable of targeting specific cells and killing such cells directly. By producing targeting proteins and toxins according to the methods of the present invention, toxin-fusion proteins normally refractory to recombinant production in eukaryotic cells, can be produced. The present invention also discloses nucleic acid constructs encoding such toxin-fusion proteins and the use of these fusion proteins in the treatment of various disorders, including proliferative disorders such as cancer.

In one embodiment, a nucleic acid construct is disclosed including, in operable linkage, nucleic acid signaling elements for homologous recombination and expression of the fusion protein in a plant or algae plastid and a first polynucleotide sequence encoding a first polypeptide and a second polynucleotide sequence encoding a toxin, where the first and second polynucleotide sequences are expressed as a fusion protein.

In one aspect, the first polynucleotide encodes a non-plastid, non-plant, eukaryotic polypeptide. In another aspect, the first polynucleotide encodes a binding domain, where the binding domain is selected from an prokaryotic cell or a binding fragment thereof, where the fragment binds to a select target, or a synthetic polypeptide comprising the binding domain of the prokaryotic cell or fragment thereof.

In one aspect, the binding domain comprises an antibody or an antigen binding fragment thereof. In another related aspect, the antibody is a complete antibody, including the binding domain of the antibody that recognizes a cell surface marker.

In one aspect, the binding domain is an Fc-region. In a related aspect, the Fc region is hIgG1Fc.

In one aspect, the cell surface marker is expressed on B-cells, including but not limited to CD19.

In another aspect, the first polynucleotide encodes mammary associated serum amyloid (SAA).

In one aspect, the toxin is functional in a eukaryotic cell, and may include, but is not limited to, an endotoxin or exotoxin. In a related aspect, the toxin is exotoxin A. In another aspect, the toxin is a toxin derived from a plant, and includes, but is not limited to, gelonin.

In one embodiment, a plant cell or algae cell or progeny thereof is disclosed which contains a construct, where the construct includes, in operable linkage, nucleic acid signaling elements for homologous recombination and expression of the fusion protein in a plant or algae plastid and a first polynucleotide sequence encoding a first polypeptide and a second polynucleotide sequence encoding a toxin, where the first and second polynucleotide sequences are expressed as a fusion protein.

In another embodiment, a plant cell or algae cell plastid is disclosed which contains a construct which includes, in operable linkage, nucleic acid signaling elements for homologous recombination and expression of the fusion protein in a plant or algae plastid and a first polynucleotide sequence a first polypeptide and a second polynucleotide sequence encoding a toxin, where the first and second polynucleotide sequences are expressed as a fusion protein.

In one aspect, the plant cell, algae cell or progeny contains the first and second polynucleotides that are stably integrated into the plastid of the cell. In another aspect, a vector includes such a construct.

In one embodiment, a method of producing a bifunctional fusion protein is disclosed, including contacting a plastid with one or more expression constructs, where the expression constructs include, in operably linkage, a nucleic acid signal element for homologous recombination and expression of the fusion protein in the plastid and a first polynucleotide sequence encoding a first polypeptide and a second polynucleotide sequence encoding a toxin, wherein the first and second polynucleotide sequences are expressed as a fusion protein, allowing the construct to integrate into the genome of the plastid, and expressing the fusion protein encoded by the construct.

In one aspect, the plastid is in a plant cell or algae cell or progeny thereof.

In another aspect, the first polynucleotide encodes an antibody or an antigen binding fragment thereof, including that the binding domain of the antibody recognizes a cell surface marker. In a related aspect, the binding domain recognizes a cell surface marker preferentially expressed on B-cells, including but not limited to, CD19.

In a related aspect, the method further includes isolating the expressed protein from the plastid.

In another aspect, the first polynucleotide encodes mammary associated serum amyloid (SAA).

In one aspect, a toxin is functional in a eukaryotic cell, and may include, but is not limited to, a cellular toxin such as single-chain bacterial toxins (e.g., Pseudomonas exotoxin, diphtheria toxin) or plant holotoxins (e.g., class II ribosome inactivating proteins such as ricin, abrin, mistletoe lectin, moceccin, or abrin) or hemitoxins (e.g., class I ribosome inactivating proteins such as gelonin, saporin, pokeweed antiviral protein, bouganin, or bryodin 1). In a related aspect, the toxin is exotoxin A. In another aspect, the toxin is a toxin derived from a plant, and includes, but is not limited to, gelonin.

In another embodiment, a plastid is disclosed which includes a nucleic acid expression construct, where the construct includes, in operable linkage, nucleic acid signaling elements for homologous recombination and expression of the fusion protein in a plant or algae plastid and a first polynucleotide sequence encoding a non-plastid, non-plant, eukaryotic polypeptide and a second polynucleotide sequence encoding a toxin, where the first and second polynucleotide sequences are expressed as a fusion protein. In a related aspect, the plastid is a chloroplast.

In one embodiment, microalgae, macroalgae or progeny thereof, contain a plastid, where the plastid includes a nucleic acid expression construct, where the construct includes, in operable linkage, nucleic acid signaling elements for homologous recombination and expression of the fusion protein in a plant or algae plastid and a first polynucleotide sequence encoding a non-plastid, non-plant, eukaryotic polypeptide and a second polynucleotide sequence encoding a toxin, where the first and second polynucleotide sequences are expressed as a fusion protein.

In one aspect, the algae is Chlamydomonas reinhardtii.

In another embodiment, an isolated fusion protein is disclosed which is generated by the steps including contacting a plastid with one or more expression constructs, where the expression constructs include, in operably linkage, a nucleic acid signal element for homologous recombination and expression of the fusion protein in the plastid and a first polynucleotide sequence encoding a first polypeptide and a second polynucleotide sequence encoding a toxin, wherein the first and second polynucleotide sequences are expressed as a fusion protein, allowing the construct to integrate into the genome of the plastid, and expressing the fusion protein encoded by the construct.

In one embodiment, a method of killing a eukaryotic cell is disclosed including contacting the eukaryotic cell with a fusion protein isolated from a plant cell or algae cell or a plant cell or algae cell plastid which contains a construct which includes, in operable linkage, nucleic acid signaling elements for homologous recombination and expression of the fusion protein in a plant or algae plastid and a first polynucleotide sequence encoding a first polypeptide and a second polynucleotide sequence encoding a toxin, where the first and second polynucleotide sequences are expressed as a fusion protein.

In another embodiment, a method of specifically inhibiting B-cell proliferation is disclosed including treating animal or human cells with a therapeutically effective dose of the fusion protein which is generated by the steps including contacting a plastid with one or more expression constructs, where the expression constructs include, in operably linkage, a nucleic acid signal element for homologous recombination and expression of the fusion protein in the plastid and a first polynucleotide sequence encoding a first polypeptide and a second polynucleotide sequence encoding a toxin, wherein the first and second polynucleotide sequences are expressed as a fusion protein, allowing the construct to integrate into the genome of the plastid, and expressing the fusion protein encoded by the construct.

### BRIEF DESCRIPTION OF THE DRAWINGS

FIG. 1 shows the amino acid sequence of the anti-CD19 single chain antibody (SEQ ID NO:1). The sequence was derived from a mouse anti-human CD19 antibody. Amino acid residues 1 to 114 define the variable regions of the light chain, amino acid residues 115 to 134 define a flexible peptide linker, amino acid residues 135 to 263 define the variable region of the heavy chain, and amino acid residues 264 to 290 define the FLAG epitope tag.

FIG. 2 shows the nucleotide and amino acid sequences of domains II and III from exotoxin A of Pseudomonas (SEQ ID NOS:2 and 3, respectively). Amino acid residues 1 to 364 define the catalytic and translocation domain II and III, while amino acid residues 365 to 391 indicate the FLAG epitope tag.

FIG. 3 shows the nucleotide and amino acid sequences of CD19 scFv-exotoxin A fusion protein (SEQ ID NOS:4 and 5, respectively). Amino acid residues 1 to 113 define the variable regions of the light chain, amino acid residues 114 to 133 and 263 to 280 define flexible peptide linkers and amino acid residues 134 to 260 define the variable region of the heavy chain. Exotoxin A domains II and III are defined by amino acid residues 281 to 644 and amino acid residues 645 to 671 define the FLAG epitope tag.

FIG. 4 shows the Southern blot analysis of C. reinhardtii transgenic lines containing, CD19 scFv, CD19-exotoxin A, and Exotoxin A. Blots were probed with a CD19 scFv cDNA (left panel), an ETA domains II and III probe (central panel), or a chloroplast genomic fragment (right panel).

FIG. 5 shows a Northern blot analysis of recombinant mRNA accumulation in three transgenic lines. Total RNA was separated on denaturing agarose gels and stained with ethidium bromide (left panel, or blotted to membranes and hybridized with D1, exotoxin A, or CD19 scFv coding region.

FIG. 6 shows a Western blot analysis of recombinant protein accumulation in C. reinhardtii transgenic lines. Total proteins from wt and transgenic lines were blotted to membranes and decorated with anti-exotoxin A (left panel) or anti-FLAG (right panel) antisera.

FIG. 7 shows an exotoxin A domain III ribosylation activity assay. Exotoxin A specifically ribosylates eukaryotic elongation factor 2 (eEF2). Equal amounts of eEF2 were incubated with bacterial expressed exotoxin A domains II and III (pET exotoxin A), or with C. reinhardtii protein extracts from wt, a transgenic line expressing CD19 scFv alone, CD19-exotoxin A fusion protein, exotoxin A domain III, or no protein. The left panel shows a stain gel of the proteins after separation by SDS-PAGE.

FIG. 8 illustrates the binding of CD19-ETA to CD19 positive B-cells. Top panel shows fluorescence of Ramos B-cells incubated with increasing concentrations of CD19-ETA-flag and a FITC labeled anti-flag antibody. The highest concentration being represented by the second line from the top of the graph with the control represented by the top most line. The lower panel shows human peripheral blood lymphocytes (PBL) labeled with the same CD19-ETA-Flag and FITC labeled anti-Flag as in the top panel. The highest concentration being shown by the bottom most line of the graph with the control represented by the top most line.

FIG. 9 shows PBL cell viability after treatment with exotoxin A alone (lines 1-3 from the bottom of the graph), CD19 antibody alone (lines 4-6 from the bottom of the graph), or CD19-ETA antibody toxin fusion (lines 7-10 from the bottom of the graph). Cells were stained with anti-annexin PE.

FIG. 10 shows the nucleotide and amino acid sequences of the SAA-nGelonin fusion protein (SEQ ID NOS:6 and 7, respectively). Amino acid residues 1 to 113 define the codon optimized bovine serum amyloid A 3 protein, amino acid residues 114 to 119 define the flexible peptide linker, amino acid residues 120 to 128 define a TEV protease site, amino acid residues 129 to 379 define native Gelonin, and amino acid residues 380 to 405 at the carboxy terminus define the FLAG epitope tag.

FIG. 11 shows a Western blot analysis of recombinant rGelonin and SAA-nGelonin protein accumulation in C. reinhardtii transgenic chloroplasts. Total proteins from wt, a transgenic line expressing rGel and a dilution series of proteins from a transgenic line expressing SAA-nGelonin are shown. The proteins were blotted to membranes and decorated with anti-FLAG (right panel) antisera.

FIG. 12 shows an in vitro activity assay of isolated chloroplast expressed SAA-nGelonin. Lane 2 shows a control primer extension product. Lane 3 shows primer extension with no added protein, lane 4 shows primer extension with bacterially expressed rGelonin added, lane 6 shows primer extension with purified SAA-nGelonin added.

FIG. 13 shows nucleotide and amino acid sequences of the native gelonin sequence linked to FLAG epitope tag (SEQ ID NOS:8 and 9, respectively). Amino acid residues 1 to 253 define native Gelonin, and amino acid residues 254 to 281 at the carboxy terminus define the FLAG epitope tag.

FIG. 14 shows the nucleotide and amino acid sequences of the CD19 scFv-Gelonin fusion protein (SEQ ID NOS:10 and 11, respectively). Amino acid residues 1 to 115 define the variable regions of the light chain, amino acid residues 116 to 135 define the flexible peptide linker, amino acid residues 136 to 264 define the variable region of the heavy chain, amino acid residues 265 to 276 define the flexible peptide linker, amino acid residues 277 to 527 define native Gelonin, and amino acid residues 528 to 556 at the carboxy terminus define the FLAG epitope tag.

FIG. 15 shows an in vitro gelonin assay using the CD19 scFv-Gelonin fusion protein. Gelonin activity is assayed by primer extension with radio-labeled primer. Yeast ribosomes were treated with purified recombinant gelonin, CD19: Gelonin, or untreated (no protein). Active gelonin will cleave the rRNA within the ricin loop. After treatment rRNA is isolated and used as a template for primer extension. ‘Experimental’ primers will give a product if gelonin activity is present (FIG. 15A). ‘Control’ primers will give a product (FIG. 15B) if rRNA is present.

FIG. 16 shows various experiments using the CD19 scFv-Gelonin fusion protein. FIG. 16A shows a Western blot of starting material, purified by FLAG affinity from crude algae lysate, before and after concentration (S1 and S2 respectively), then elutions from desalting column. FIG. 16B shows the elution profile from desalting column. Darker line shows UV absorbance, lighter line shows conductivity (salt). FIG. 16C shows a Western blot of purified desalted samples. Elutions 2-10 from desalting column were pooled (lane 1) and concentrated (lane 2), and filtered (lane 4).

FIG. 17 shows the nucleotide and amino acid sequences of the CD19 scFv-CH2-ETA fusion protein (SEQ ID NOS:12 and 13, respectively). Amino acid residues 1 to 261 define the variable regions of the light chain, amino acid residues 262 to 381 define the CH2 constant domain, amino acid residues 382 to 772 define Exotoxin A, amino acid residues 773 to 780 define a TEV cleavage site, amino acid residues 781 to 786 define the flexible peptide linker, and amino acid residues 782 to 791 at the carboxy terminus define the FLAG epitope tag.

FIG. 18 shows expression of an anti-CD19-scFv-heavy chain CH2 domain-exotoxin A chimeric protein. Four transgenic lines, 32-1, 34-3, 41-4 and 45-1 were analyzed by western blot analysis for the accumulation of the chimeric protein. Protein from non-transformed wild type cells (Wt) was loaded in Lane 1. The chimeric antibody-toxin protein (arrowhead) accumulates as a soluble protein at the correct molecular weight (85 kD) in at least three of the transgenic lines, 32-1, 41-4 and 45-1. The chimeric protein was visualized using an anti-ETA antibody.

### DETAILED DESCRIPTION OF THE INVENTION

Before the present composition, methods, and treatment methodology are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention discloses recombinant proteins containing a genetic fusion between a first protein or peptide and a protein toxin or peptide toxin, where such a fusion protein is produced in a eukaryotic cell and would normally be lethal to such cells. The recombinant method does not require modifying the toxin or nucleic acid sequence encoding the toxin to alter toxin activity. In one embodiment, a disclosed fusion protein comprises an immunoglobulin binding domain, including but not limited to, an anti-CD19 single chain antibody (CD19) and a bacterial protein, including but not limited to, exotoxin A protein (ETA) of Pseudomonas. In one aspect, the a CD19-ETA fusion protein gene may be transformed into the chloroplast of a plant cell, including but not limited to, the green algae C. reinhardtii and a bioactive antibody-toxin may be produced in eukaryotic cell organelles (e.g., chloroplasts). In another aspect, the purified CD19-ETA is cytotoxic to CD19 positive Ramos human cell line, as well as cytotoxic to activated peripheral blood lymphocytes, in vitro.

In another embodiment, the protein is a lipid transporter, including but not limited to, serum amyloid A3 (SAA) and a plant derived protein toxin or peptide toxin, including but not limited to, gelonin or ricin.

Data is provided that shows that eukaryotic toxins can be expressed in eukaryotic cells if the toxin is produced within a subcellular organelle, like the chloroplast. These data also demonstrate the utility of plants, including but not limited to, green algae, for the production of complex multi-domain proteins as soluble bioactive therapeutic agents.

As used herein “cognate” is used in a comparative sense to refer to genetic elements that are typically associated with a specific reference gene. For example, for the Photosystem II (PSII) gene psbA (i.e., a specific reference gene), cognate genetic elements would include, but are not limited to, a psbA promoter, psbA 5′ UTR, and psbA 3′ UTR. Contrapositively, “non-cognate” would refer to genetic elements that are not typically related to a specific reference gene. For example, but not limited to, where a chimeric construct comprising a psbA promoter and psbD 5′ UTR is to be homologously recombined at a psbA site, the 5′ UTR in the construct would be non-cognate to psbA.

As used herein “nucleic acid signaling element” is used broadly herein to refer to a nucleotide sequence that regulates the transcription or translation of a polynucleotide or the localization of a polypeptide to which it is operatively linked. A nucleic acid signaling element can be a promoter, enhancer, transcription terminator, an initiation (start) codon, a splicing signal for intron excision and maintenance of a correct reading frame, a STOP codon, an amber or ochre codon, an IRES, an RBS, a sequence encoding a protein intron (intein) acceptor or donor splice site, or a sequence that targets a polypeptide to a particular location, for example, a cell compartmentalization signal, which can be useful for targeting a polypeptide to the cytosol, nucleus, plasma membrane, endoplasmic reticulum, mitochondrial membrane or matrix, chloroplast membrane or lumen, medial trans-Golgi cisternae, or a lysosome or endosome. Cell compartmentalization domains are well known in the art and include, for example, a peptide containing amino acid residues 1 to 81 of human type II membrane-anchored protein galactosyltransferase, the chloroplast targeting domain from the nuclear-encoded small subunit of plant ribulose bisphosphate carboxylase, or amino acid residues 1 to 12 of the presequence of subunit IV of cytochrome c oxidase (see, also, Hancock et al., EMBO J. 10:4033-4039, 1991; Buss et al., Mol. Cell. Biol. 8:3960-3963, 1988; U.S. Pat. No. 5,776,689). Inclusion of a cell compartmentalization domain in a polypeptide produced using a method of the invention can allow use of the polypeptide, which can comprise a protein complex, where it is desired to target the polypeptide to a particular cellular compartment in a cell.

As used herein “binding domain” means a region of a protein or peptide which allows for stereoselective, specific interaction with a ligand, substrate, epitope, antigen, cell surface markers, cell surface receptors, and the like, and includes, but is not limited to, antibodies, receptors, hormones, cytokines, chemokines, interferons, and fragments thereof.

As used herein “cell surface markers” means a polypeptide, carbohydrate, lipid or a combination thereof on the plasma surface of a cell. In one embodiment, such markers include clusters of differentiation (CD), including, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, and the like. In one embodiment, the CD is specific for B-cells. In a related aspect, the marker is CD19.

As used herein “toxin” includes bacterial and plant derived toxins. For example, such toxins are proteins or peptides, and include botulism toxin, tetanus toxin, shigella neurotoxin, diphtheria toxin, hemolysins, leukocidins, anthrax toxin, adenylate cyclase toxin, cholera enterotoxin, E. coli LT toxin, E. coli ST toxin, exotoxins, shiga toxin, perfringens toxin, exotoxin A, pertussis toxin, toxic shock syndrome toxin, exfoliatin toxin, erythrogenic toxin, and the like. In one aspect, the toxin is endotoxin A. In another aspect, plant toxins include single chain ribosome inactivating proteins. In one aspect, proteinacious plant toxins are disclosed, including, but not limited to, gelonin and ricin. In another aspect, “obtained from a plant” means isolated, extracted or a polypeptide/peptide/protein which is normally expressed by a plant that is produced either synthetically or recombinantly.

As used herein “multifunctional” means having at least two functions. For example, a fusion protein comprising a binding domain and a toxin domain would be bifunctional.

As used herein “progeny” means a descendant or offspring, as a child, plant or animal. For example, daughter cells from a transgenic algae are progeny of the transgenic algae.

As used herein “transgene” means any gene carried by a vector or vehicle, where the vector or vehicle includes, but is not limited to, plasmids and viral vectors.

In a related aspect, integration of chimeric constructs into plastid genomes includes homologous recombination. In a further related aspect, cells transformed by the methods of the present invention may be homoplasmic or heteroplasmic for the integration, wherein homoplastic means all copies of the transformed plastid genome carry the same chimeric construct.

As used herein, the term “modulate” refers to a qualitative or quantitative increase or decrease in the amount of an expressed gene product. For example, where the use of light increases or decreases the measured amount of protein or RNA expressed by a cell, such light modulates the expression of that protein or RNA. In one aspect, modulation of expression includes autoregulation, where “autoregulation” refers to processes that maintain a generally constant physiological state in a cell or organism, and includes autorepression and autoinduction.

In a related aspect, autorepression is a process by which excess endogenous protein or endogenous mRNA results in decreasing the amount of expression of that endogenous protein. In a further related aspect, reduction of endogenous protein synthesis will result in increased transgene expression. In one aspect, operatively linking non-cognate genetic elements (e.g., promoters) to the endogenous gene is used to drive low levels of endogenous protein expression. In another aspect, mutations are introduced into the endogenous gene sequence and/or cognate genetic elements to reduce expression of the endogenous protein.

As used herein, the term “multiple cloning site” is used broadly to refer to any nucleotide or nucleotide sequence that facilitates linkage of a first polynucleotide to a second polynucleotide. Generally, a cloning site comprises one or a plurality of restriction endonuclease recognition sites, for example, a cloning site, or one or a plurality of recombinase recognition sites, for example, a loxP site or an att site, or a combination of such sites. The cloning site can be provided to facilitate insertion or linkage, which can be operative linkage, of the first and second polynucleotide, for example, a first polynucleotide encoding a first 5′ UTR operatively linked to second polynucleotide comprising a homologous coding sequence encoding a polypeptide of interest, linked to a first 3′ UTR, which is to be translated in a prokaryote or a chloroplast or both.

In one embodiment, a chimeric construct is disclosed including a PSII reaction center protein gene promoter, PSII gene 5′ UTR, a multiple cloning site (MCS), and a PSII gene 3′ UTR, having the configuration:

PSII gene promoter-PSII gene 5′ UTR-MCS-PSII gene 3′ UTR.

In a related aspect, the PSII gene UTRs are from different PSII genes and may include, but are not limited to, a psbD 5′ UTR and a psbA 5′ UTR.

In another related aspect, the PSII gene promoter is a psbA or psbD promoter and the 3′ UTR is a psbA 3′ UTR.

In one aspect, the PSII gene promoter and PSII gene 5′ UTR are from psbD. In another aspect, the PSII gene 3′ UTR is a psbA 3′ UTR.

As used herein, the term “Photosystem 11 reaction center” refers to an intrinsic membrane-protein complex in the chloroplast made of D1 (psbA gene), D2 (psbD gene), alpha and beta subunits of cytochrome b-559 (psbE and psbF genes respectively), the psbI gene product and a few low molecular weight proteins (e.g., 9 kDa peptide [psbH gene] and 6.5 kDa peptide [psbW gene]). In a related aspect, endogenous genes embrace chloroplast genes that exhibit autoregulation of translation, and include, but are not limited to, cytochrome f (i.e., C-terminal domain) and photosystem I reaction center genes (e.g., psaA, PsaB, PsaC, PsaJ).

As used herein, the term “operatively linked” means that two or more molecules are positioned with respect to each other such that they act as a single unit and effect a function attributable to one or both molecules or a combination thereof. For example, a polynucleotide encoding a polypeptide can be operatively linked to a transcriptional or translational regulatory element, in which case the element confers its regulatory effect on the polynucleotide similarly to the way in which the regulatory element would effect a polynucleotide sequence with which it normally is associated with in a cell.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition, except that the term “synthetic polynucleotide” as used herein refers to a polynucleotide that has been modified to reflect chloroplast codon usage.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

The term “recombinant nucleic acid molecule” is used herein to refer to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide, or can comprise an encoding nucleotide sequence and a regulatory element, particularly a PSII promoter operatively linked to a PSII 5′ UTR. A recombinant nucleic acid molecule also can be based on, but manipulated so as to be different, from a naturally occurring polynucleotide, for example, a polynucleotide having one or more nucleotide changes such that a first codon, which normally is found in the polynucleotide, is biased for chloroplast codon usage, or such that a sequence of interest is introduced into the polynucleotide, for example, a restriction endonuclease recognition site or a splice site, a promoter, a DNA origin of replication, or the like.

One or more codons of an encoding polynucleotide can be biased to reflect chloroplast codon usage. Most amino acids are encoded by two or more different (degenerate) codons, and it is well recognized that various organisms utilize certain codons in preference to others. Such preferential codon usage, which also is utilized in chloroplasts, is referred to herein as “chloroplast codon usage”. Table 1 (below) shows the chloroplast codon usage for C. reinhardtii.

TABLE 1
Chloroplast Codon Usage for C. reinhardtii.
Chloroplast Codon Usage in Chlamydomonas reinhardtii

| | | | |
|---|---|---|---|
| UUU 34.1*(348**) | UCU 19.4(198) | UAU 23.7(242) | UGU 8.5(87) |
| UUC 14.2(145) | UCC 4.9(50) | UAC 10.4(106) | UGC 2.6(27) |
| UUA 72.8(742) | UCA 20.4(208) | UAA 2.7(28) | UGA 0.1(1) |
| UUG 5.6(57) | UCG 5.2(53) | UAG 0.7(7) | UGG 13.7(140) |
| CUU 14.8(151) | CCU 14.9(152) | CAU 11.1(113) | CGU 25.5(260) |
| CUC 1.0(10) | CCC 5.4(55) | CAC 8.4(86) | CGC 5.1(52) |
| CUA 6.8(69) | CCA 19.3(197) | CAA 34.8(355) | CGA 3.8(39) |
| CUG 7.2(73) | CCG 3.0(31) | CAG 5.4(55) | CGG 0.5(5) |
| AUU 44.6(455) | ACU 23.3(237) | AAU 44.0(449) | AGU 16.9(172) |
| AUC 9.7(99) | ACC 7.8(80) | AAC 19.7(201) | AGC 6.7(68) |
| AUA 8.2(84) | ACA 29.3(299) | AAA 61.5(627) | AGA 5.0(51) |
| AUG 23.3(238) | ACG 4.2(43) | AAG 11.0(112) | AGG 1.5(15) |
| GUU 27.5(280) | GCU 30.6(312) | GAU 23.8(243) | GGU 40.0(408) |
| GUC 4.6(47) | GCC 11.1(113) | GAC 11.6(118) | GGC 8.7(89) |
| GUA 26.4(269) | GCA 19.9(203) | GAA 40.3(411) | GGA 9.6(98) |
| GUG 7.1(72) | GCG 4.3(44) | GAG 6.9(70) | GGG 4.3(44) |

*Frequency of codon usage per 1,000 codons.
**Number of times observed in 36 chloroplast coding sequences (10,193 codons).

The term “biased”, when used in reference to a codon, means that the sequence of a codon in a polynucleotide has been changed such that the codon is one that is used preferentially in chloroplasts (see Table 1). A polynucleotide that is biased for chloroplast codon usage can be synthesized de novo, or can be genetically modified using routine recombinant DNA techniques, for example, by a site directed mutagenesis method, to change one or more codons such that they are biased for chloroplast codon usage. As disclosed herein, chloroplast codon bias can be variously skewed in different plants, including, for example, in alga chloroplasts as compared to tobacco.

Table 1 exemplifies codons that are preferentially used in alga chloroplast genes. The term “chloroplast codon usage” is used herein to refer to such codons, and is used in a comparative sense with respect to degenerate codons that encode the same amino acid but are less likely to be found as a codon in a chloroplast gene. The term “biased”, when used in reference to chloroplast codon usage, refers to the manipulation of a polynucleotide such that one or more nucleotides of one or more codons is changed, resulting in a codon that is preferentially used in chloroplasts. Chloroplast codon bias is exemplified herein by the alga chloroplast codon bias as set forth in Table 1. The chloroplast codon bias can, but need not, be selected based on a particular plant in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect chloroplast codon usage, or can be the de novo synthesis of polynucleotide sequence such that the change (bias) is introduced as a consequence of the synthesis procedure.

In addition to utilizing chloroplast codon bias as a means to provide efficient translation of a polypeptide, it will be recognized that an alternative means for obtaining efficient translation of a polypeptide in a chloroplast to re-engineer the chloroplast genome (e.g., a C. reinhardtii chloroplast genome) for the expression of tRNAs not otherwise expressed in the chloroplast genome. Such an engineered algae expressing one or more heterologous tRNA molecules provides the advantage that it would obviate a requirement to modify every polynucleotide of interest that is to be introduced into and expressed from a chloroplast genome; instead, algae such as C. reinhardtii that comprise a genetically modified chloroplast genome can be provided and utilized for efficient translation of a polypeptide according to a method of the invention. Correlations between tRNA abundance and codon usage in highly expressed genes is well known in the art. In E. coli, for example, re-engineering of strains to express underutilized tRNAs has been shown to result in enhanced expression of genes which utilize these codons. Utilizing endogenous tRNA genes, site directed mutagenesis can be used to make a synthetic tRNA gene, which can be introduced into chloroplasts to complement rare or unused tRNA genes in a chloroplast genome such as a C. reinhardtii chloroplast genome.

Generally, the chloroplast codon bias selected for purposes of the present invention, including, for example, in preparing a synthetic polynucleotide as disclosed herein reflects chloroplast codon usage of a plant chloroplast, and includes a codon bias that, with respect to the third position of a codon, is skewed towards A/T, for example, where the third position has greater than about 66% AT bias, particularly greater than about 70% AT bias. As such, chloroplast codon biased for purposes of the present invention excludes the third position bias observed, for example, in Nicotiana tabacus (tobacco), shown to have 34.56% GC bias in the third codon position. In one embodiment, the chloroplast codon usage is biased to reflect alga chloroplast codon usage, for example, C. reinhardtii, which has about 74.6% AT bias in the third codon position.

In one embodiment, a method to produce multifunctional fusion polypeptides/proteins is disclosed. The term “polypeptides/protein” is used broadly to refer to macromolecules comprising linear polymers of amino acids which act in biological systems, for example, as structural components, enzymes, chemical messengers, receptors, ligands, regulators, hormones, and the like. In one aspect, a plant cell or algae cell or progeny thereof is disclosed which contains a construct, where the construct includes, in operable linkage, nucleic acid signaling elements for homologous recombination and expression of the bifunctional fusion protein in a plant or algae plastid and a first polynucleotide sequence encoding a first polypeptide and a second polynucleotide sequence encoding a toxin, where the first and second polynucleotide sequences are expressed as a fusion protein. In another aspect, the fusion protein may include stabilizing molecules or domains, such as Fc domains and low complexity linkers. Such stabilizing molecules may form tripartite structures, which include a stabilizing domain-targeting domain-toxin domain. In one aspect, a fusion protein may comprise one or more stabilizing domains. Such tripartite molecules may also contain a small molecule drug, including, but not limited to therapeutic compounds. In one aspect, the tripartite molecule may comprise a purification domain (e.g., but not limited to, a His₆ (SEQ ID NO:14) or FLAG tag).

In a related aspect, such tripartite molecules may be encoded by a single polynucleotide. In another aspect, a functional binding domain of the tripartite molecule may comprise multimers of subunits to form a multimeric complex, where the tripartite structure is encoded with a first subunit of a multimer. The second or third or more subunits of the multimeric complex may be encoded on separate polynucleotides. In one aspect, the second, third or more subunits are integrated into different sites in the chloroplast genome, where each integrated subunit encoding polynucleotide comprises separate recombinational targeting sequences, promoters/5′ UTR regulatory sequences, and 3′ UTR sequences. In one aspect, the multimeric complex comprises a heavy chain and a light chain of an complete antibody.

In one embodiment, such fusion protein comprise multiple binding domains for targeting multiple surface markers. In one aspect, the fusion protein includes one or more binding domains which specifically target CD19, CD20, and CD21. In other aspects, other clusters of differentiation (CD) may include, but are not limited to, CD 1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD22, CD23, Cd24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49aq, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, and the like.

In another aspect, such polypeptides/proteins would include functional protein complexes, such as antibodies. The term “antibody” is used broadly herein to refer to a polypeptide or a protein complex that can specifically bind an epitope of an antigen. As used in this invention, the term “epitope” refers to an antigenic determinant on an antigen, such as a cell surface marker, to which the paratope of an antibody, such as an CD19 specific antibody, binds. Antigenic determinants usually consist of chemically active surface groupings of molecules, such as amino acids or sugar side chains, and can have specific three dimensional structural characteristics, as well as specific charge characteristics.

Generally, an antibody contains at least one antigen binding domain that is formed by an association of a heavy chain variable region domain and a light chain variable region domain, particularly the hypervariable regions. An antibody generated according to a method of the invention can be based on naturally occurring antibodies, for example, bivalent antibodies, which contain two antigen binding domains formed by first heavy and light chain variable regions and second heavy and light chain variable regions (e.g., an IgG or IgA isotype) or by a first heavy chain variable region and a second heavy chain variable region (V_{HH} antibodies), or on non-naturally occurring antibodies, including, for example, single chain antibodies, chimeric antibodies, bifunctional antibodies, and humanized antibodies, as well as antigen-binding fragments of an antibody, for example, an Fab fragment, an Fd fragment, an Fv fragment, and the like. In a related aspect, a heterologous gene encodes a single chain antibody comprising a heavy chain operatively linked to a light chain.

Antigens that can be used in the present invention specific antibodies select polypeptides or polypeptide fragments. The polypeptide or peptide used to immunize an animal can be obtained by standard recombinant, chemical synthetic, or purification methods. As is well known in the art, in order to increase immunogenicity, an antigen can be conjugated to a carrier protein. Commonly used carriers include keyhole limpet hemocyanin (KLH), thyroglobulin, bovine serum albumin (BSA), and tetanus toxoid. The coupled peptide is then used to immunize the animal (e.g., a mouse, a rat, or a rabbit). In addition to such carriers, well known adjuvants can be administered with the antigen to facilitate induction of a strong immune response.

In another related aspect, polynucleotides useful for practicing a method of the producing such antibodies can be isolated from cells producing the antibodies of interest, for example, B cells from an immunized subject or from an individual exposed to a particular antigen, can be synthesized de novo using well known methods of polynucleotide synthesis, can be produced recombinantly or can be obtained, for example, by screening combinatorial libraries of polynucleotides that encode variable heavy chains and variable light chains and can be biased for chloroplast codon usage, if desired (see Table 1). These and other methods of making polynucleotides encoding, for example, chimeric, humanized, CDR-grafted, single chain, and bifunctional antibodies are well known to those skilled in the art.

Polynucleotides encoding humanized monoclonal antibodies, for example, can be obtained by transferring nucleotide sequences encoding mouse complementarity determining regions (CDRs) from heavy and light variable chains of the mouse immunoglobulin gene into a human variable domain gene, and then substituting human residues in the framework regions of the murine counterparts. General techniques for cloning murine immunoglobulin variable domains are known is the art, as well as methods for producing humanized monoclonal antibodies.

The disclosed methods can also be practiced using polynucleotides encoding human antibody fragments isolated from a combinatorial immunoglobulin library. Cloning and expression vectors that are useful for producing a human immunoglobulin phage library can be obtained, for example, from Stratagene Cloning Systems (La Jolla, Calif.).

A polynucleotide encoding a human monoclonal antibody also can be obtained, for example, from transgenic mice that have been engineered to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas, from which polynucleotides useful for practicing a method of the invention can be obtained. Methods for obtaining human antibodies from transgenic mice have been previously described, and such transgenic mice are commercially available (e.g., Abgenix, Inc.; Fremont Calif.).

Monoclonal antibodies used in the method of the invention are suited for use, for example, in immunoassays in which they can be utilized in liquid phase or bound to a solid phase carrier. In addition, the monoclonal antibodies in these immunoassays can be detectably labeled in various ways. Examples of types of immunoassays which can utilize monoclonal antibodies of the invention are competitive and non-competitive immunoassays in either a direct or indirect format. Examples of such immunoassays are the radioimmunoassay (RIA) and the sandwich (immunometric) assay. Detection of the antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The term “immunometric assay” or “sandwich immunoassay”, includes simultaneous sandwich, forward sandwich and reverse sandwich immunoassays. These terms are well understood by those skilled in the art. Those of skill will also appreciate that antibodies according to the present invention will be useful in other variations and forms of assays which are presently known or which may be developed in the future. These are intended to be included within the scope of the present invention.

Monoclonal antibodies of the present invention may also be bound to many different carriers. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such using routine experimentation.

In performing the assays it may be desirable to include certain “blockers” in the incubation medium (usually added with the labeled soluble antibody). The “blockers” are added to assure that non-specific proteins present in the experimental sample do not cross-link or destroy the antibodies on the solid phase support, or the radiolabeled indicator antibody, to yield false positive or false negative results. The selection of “blockers” therefore may add substantially to the specificity of the assays described in the present invention.

It has been found that a number of nonrelevant (i.e., nonspecific) antibodies of the same class or subclass (isotype) as those used in the assays (e.g. IgG1, IgG2a, IgM, etc.) can be used as “blockers”. The concentration of the “blockers” (normally 1-100 μg/μl) may be important, in order to maintain the proper sensitivity yet inhibit any unwanted interference by mutually occurring cross reactive proteins in the specimen.

In using a monoclonal antibody for the in vivo detection of antigen, the detectably labeled monoclonal antibody is given in a dose which is diagnostically effective. The term “diagnostically effective” means that the amount of detectably labeled monoclonal antibody is administered in sufficient quantity to enable detection of the site having the antigen of interest for which the monoclonal antibodies are specific. The concentration of detectably labeled monoclonal antibody which is administered should be sufficient such that the binding to those antigens/epitopes of interest is detectable compared to the background. Further, it is desirable that the detectably labeled monoclonal antibody be rapidly cleared from the circulatory system in order to give the best target-to-background signal ratio.

As a rule, the dosage of detectably labeled monoclonal antibody for in vivo diagnosis will vary depending on such factors as age, sex, and extent of disease of the individual. The dosage of monoclonal antibody can vary from about 0.001 mg/m² to about 500 mg/m², preferably 0.1 mg/m² to about 200 mg/m², most preferably about 0.1 mg/m² to about 10 mg/m². Such dosages may vary, for example, depending on whether multiple injections are given, tumor burden, and other factors known to those of skill in the art.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of photons in the 140-250 keV range, which may be readily detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr, and ²⁰¹Ti.

A monoclonal antibody useful in the method of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ¹⁵⁷Gd, ⁵⁵Mn, ¹⁶²Dy, ⁵²Cr, and ⁵⁶Fe.

The polynucleotide also can be one encoding an antigen binding fragment of an antibody. Antigen binding antibody fragments, which include, for example, Fv, Fab, Fab′, Fd, and F(ab′)₂ fragments, are well known in the art, and were originally identified by proteolytic hydrolysis of antibodies. For example, antibody fragments can be obtained by pepsin or papain digestion of whole antibodies by conventional methods. Antibody fragments produced by enzymatic cleavage of antibodies with pepsin generate a 5S fragment denoted F(ab′)₂. This fragment can be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin may produce two monovalent Fab′ fragments and an Fc fragment directly.

Another form of an antibody fragment is a peptide coding for a single complementarity-determining region (CDR). CDR peptides can be obtained by constructing a polynucleotide encoding the CDR of an antibody of interest, for example, by using the polymerase chain reaction to synthesize the variable region from RNA of antibody-producing cells. Polynucleotides encoding such antibody fragments, including subunits of such fragments and peptide linkers joining, for example, a heavy chain variable region and light chain variable region, can be prepared by chemical synthesis methods or using routine recombinant DNA methods, beginning with polynucleotides encoding full length heavy chains and light chains, which can be obtained as described above.

The antibodies of the present invention can also include single chain antibodies (“SCA”). These antibodies are genetically engineered single chain molecules containing the variable region of a light chain and the variable region of a heavy chain, linked by a suitable, flexible polypeptide linker.

As an alternative to full length antibodies, including monoclonal antibodies, an equally viable approach utilizes toxins fused to Fc regions (typically hinge, C_{H}2-C_{H}3 domains of heavy chain hIgG1, 2, 3, 4 or IgA, IgE, IgM or IgD molecules) of monoclonal antibodies. These Fc regions may be native, or modified in ways that increase or decrease their affinity with specific Fc receptors. For example, modifications to the Fc region of hIgG1 molecules can increase their interaction with FcγRIII on effector cells, thereby modulating ADCC. Likewise, modifications to Fc regions on hIgG1 can impact their interactions with FcγRIIB, the inhibitory Fc receptor, on effector cells, again to modulate ADCC or to kill a particular population of cells when fused to toxins of the present invention.

The Fc region allows antibodies to activate the immune system, and is selective/specific for antibody isotype. For example, in IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. In one aspect, the Fc region is hIgG1Fc.

The Fc portion of these molecules imparts increased half life to the toxins to which they are fused through their increased size and provides a standardized and potentially modifiable means of purification via Protein A or G affinity chromatography.

Another application of Fc fusion proteins as disclosed is for increasing the potency of the toxins which are fused to Fc regions. While not being bound by theory, this increase in potency may be conferred by several mechanisms, including, but not limited to, increasing molecular weight leading to oligomerization. Such oligomerization can result in decreased loss of the toxin via renal filtration. In one embodiment, a nucleic acid construct is disclosed including, in operable linkage, nucleic acid signaling elements for homologous recombination and expression of the fusion protein in a plant or algae plastid and a first polynucleotide sequence encoding a first polypeptide and a second polynucleotide sequence encoding a toxin.

In one aspect, the first polynucleotide encodes an Fc region, or fragment thereof, where the Fc region, is a protein that mediates different immuological effects including, but not limited to, opsonization, cell lysis, and degranulation of mast cells, basophils and eosinophils.

IgG exhibits the highest synthetic rate and longest biological half-life of any immunoglobulin in serum. Complement activation is possibly the most important biological function of IgG. Activation of the complement cascade by the classical pathway is initiated by binding of C1 to sites on the Fc portion of human IgG. Another vital function of the human IgG is its ability to bind to cell surface Fc receptors. Once it is fixed to the surface of certain cell types, the IgG antibody can complex antigen and facilitate clearance of antigens or immune complexes by phagocytosis. Three classes of human IgG Fc receptors (FcR) on leukocytes have been reported: the FcR-I, FcR-II, and low affinity receptor [FcR-lo]. These are distinguished by their presence on different cell types, by their molecular weights and by their differential abilities to bind untreated or aggregated IgG myeloma protein of the four subclasses. These receptors are expressed differentially on overlapping populations of leukocytes: FcR-I on monocytes; FcR-II on monocytes neutrophils, eosinophils, platelets, and B cells; and FcR-lo on neutrophils, macrophages, and killer T cells.

In one embodiment, a nucleic acid construct is disclosed including, in operable linkage, nucleic acid signaling elements for homologous recombination and expression of the fusion protein in a plant or algae plastid and a first polynucleotide sequence encoding a polypeptide consisting essentially of an Fc region and a second polynucleotide sequence encoding a toxin.

In one aspect, a toxin is functional in a eukaryotic cell, and may include, but is not limited to, a cellular toxin such as single-chain bacterial toxins (e.g., Pseudomonas exotoxin, diphtheria toxin) or plant holotoxins (e.g., class II ribosome inactivating proteins such as ricin, abrin, mistletoe lectin, moceccin, or abrin) or hemitoxins (e.g., class I ribosome inactivating proteins such as gelonin, saporin, pokeweed antiviral protein, bouganin, or bryodin 1). In a related aspect, the toxin is exotoxin A. In another aspect, the toxin is a toxin derived from a plant, and includes, but is not limited to, gelonin.

Single celled alga, like C. reinhardtii, are essentially water borne plants and as such can produce proteins in a very cost effective manner. In addition, algae can be grown in complete containment, and there are a number of companies around the world that have develop large scale production of algae as human nutraceuticals or as a food source for farmed fish and other organisms. Capitalization costs for an algal production facility is also much less costly than for other types of cell culture, mainly because of the nature of algae and it's ability to grow with minimal input, using CO₂ as a carbon source and sunlight as an energy source. Although in many ways algae are an ideal system for therapeutic protein production there are a number of technical challenges that need to be met before algae can be used as an efficient production platform. Among these challenges are developing vectors that allow for consistent high levels of protein expression.

A recombinant nucleic acid molecule useful in a method of the invention can be contained in a vector. The vector can be any vector useful for introducing a polynucleotide into a chloroplast and, preferably, includes a nucleotide sequence of chloroplast genomic DNA that is sufficient to undergo homologous recombination with chloroplast genomic DNA, for example, a nucleotide sequence comprising about 400 to 1500 or more substantially contiguous nucleotides of chloroplast genomic DNA. A number of chloroplast vectors and methods for selecting regions of a chloroplast genome for use as a vector have been described.

The entire chloroplast genome of C. reinhardtii has been sequenced (Maul et al., Plant Cell (2002) 14(11):2659-79; GenBank Acc. No. BK000554). Generally, the nucleotide sequence of the chloroplast genomic DNA is selected such that it is not a portion of a gene, including a regulatory sequence or coding sequence, particularly a gene that, if disrupted due to the homologous recombination event, would produce a deleterious effect with respect to the chloroplast, for example, for replication of the chloroplast genome, or to a plant cell containing the chloroplast. In this respect, the Accession No. disclosing the C. reinhardtii chloroplast genome sequence also provides maps showing coding and non-coding regions of the chloroplast genome, thus facilitating selection of a sequence useful for constructing a vector of the invention. For example, the chloroplast vector, p322, which is used in experiments disclosed herein, is a clone extending from the Eco (Eco RI) site at about position 143.1 kb to the Xho (Xho I) site at about position 148.5 kb.

The vector also can contain any additional nucleotide sequences that facilitate use or manipulation of the vector, for example, one or more transcriptional regulatory elements, a sequence encoding a selectable markers, one or more cloning sites, and the like. In one embodiment, the chloroplast vector contains a prokaryote origin of replication (ori), for example, an E. coli ori, thus providing a shuttle vector that can be passaged and manipulated in a prokaryote host cell as well as in a chloroplast.

The methods of the present invention are exemplified using the microalga, C. reinhardtii. The use of microalgae to express a polypeptide or protein complex according to a method of the invention provides the advantage that large populations of the microalgae can be grown, including commercially (Cyanotech Corp.; Kailua-Kona Hi.), thus allowing for production and, if desired, isolation of large amounts of a desired product. However, the ability to express, for example, functional mammalian polypeptides, including protein complexes, in the chloroplasts of any plant allows for production of crops of such plants and, therefore, the ability to conveniently produce large amounts of the polypeptides.

In one embodiment, a method of expressing a chimeric gene is disclosed including transforming an algae cell by replacing an endogenous chloroplast gene via integration of a chimeric construct having a heterologous coding sequence, a promoter sequence, and at least one UTR, wherein the promoter is cognate or non-cognate to the endogenous chloroplast gene, and cultivating the transformed algae cell. In one aspect, a gene product encoded by the heterologous coding sequence is constitutively expressed. In a related aspect, the cells are homoplasmic for the integration.

In another embodiment, a method of expressing a chimeric gene includes transforming an algae cell by replacing psbA via integration of a chimeric construct comprising a nucleic acid sequence encoding a fusion protein of the present invention, such as those set forth in SEQ ID NO:5, SEQ ID NO:7, SEQ ID NO:11 or SEQ ID NO:13, a promoter sequence, and one or more UTRS, where the promoter is cognate or non-cognate to the endogenous chloroplast gene, and cultivating the transformed algae cell. In one aspect, at least two UTRs are psbA and psbD UTRs. In a related aspect, the nucleic acid sequence (e.g., SEQ ID NO:4, SEQ ID NO:6, SEQ ID NO:10, or SEQ ID NO: 12) is driven by a psbA or other promoter.

In one embodiment, an algae cell transformed by the methods of the invention is disclosed, where the algae includes, but is not limited to, Chlamydomonas reinhardtii.

Accordingly, the methods of the invention can be practiced using any plant having chloroplasts, including, for example, macroalgae, for example, marine algae and seaweeds, as well as plants that grow in soil, for example, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals such as azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum are also included. Additional ornamentals useful for practicing a method of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Leguminous plants useful for practicing a method of the invention include beans and peas. Beans include guar, locust bean, fenugreek, soybean, garden beans, cowpea, mung bean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop. Other plants useful in the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, chicory, groundnut and zucchini.

A method of the invention can generate a plant containing chloroplasts that are genetically modified to contain a stably integrated polynucleotide. The integrated polynucleotide can comprise, for example, an encoding polynucleotide operatively linked to a first and second UTR as defined herein. Accordingly, the present invention further provides a transgenic (transplastomic) plant, which comprises one or more chloroplasts containing a polynucleotide encoding one or more heterologous polypeptides, including polypeptides that can specifically associate to form a functional protein complex.

The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

A method of producing a heterologous polypeptide or protein complex in a chloroplast or in a transgenic plant of the invention can further include a step of isolating an expressed polypeptide or protein complex from the plant cell chloroplasts. As used herein, the term “isolated” or “substantially purified” means that a polypeptide or polynucleotide being referred to is in a form that is relatively free of proteins, nucleic acids, lipids, carbohydrates or other materials with which it is naturally associated. Generally, an isolated polypeptide (or polynucleotide) constitutes at least twenty percent of a sample, and usually constitutes at least about fifty percent of a sample, particularly at least about eighty percent of a sample, and more particularly about ninety percent or ninety-five percent or more of a sample.

In one embodiment, an algae extract obtained from an algae cell transformed by replacing an endogenous chloroplast gene via integration of a chimeric construct having a heterologous coding sequence, a promoter sequence, and one or more UTRs, where the promoter is cognate or non-cognate to the endogenous chloroplast gene is disclosed.

The term “heterologous” is used herein in a comparative sense to indicate that a nucleotide sequence (or polypeptide) being referred to is from a source other than a reference source, or is linked to a second nucleotide sequence (or polypeptide) with which it is not normally associated, or is modified such that it is in a form that is not normally associated with a reference material. For example, a polynucleotide encoding an antibody is heterologous with respect to a nucleotide sequence of a plant chloroplast, as are the components of a recombinant nucleic acid molecule comprising, for example, a first nucleotide sequence operatively linked to a second nucleotide sequence, and is a polynucleotide introduced into a chloroplast where the polynucleotide is not normally found in the chloroplast.

The chloroplasts of higher plants and algae likely originated by an endosymbiotic incorporation of a photosynthetic prokaryote into a eukaryotic host. During the integration process genes were transferred from the chloroplast to the host nucleus. As such, proper photosynthetic function in the chloroplast requires both nuclear encoded proteins and plastid encoded proteins, as well as coordination of gene expression between the two genomes. Expression of nuclear and chloroplast encoded genes in plants is acutely coordinated in response to developmental and environmental factors.

In chloroplasts, regulation of gene expression generally occurs after transcription, and often during translation initiation. This regulation has been shown to be dependent upon the chloroplast translational apparatus, as well as nuclear-encoded regulatory factors. The chloroplast translational apparatus generally resembles that in bacteria; chloroplasts contain 70S ribosomes; have mRNAs that lack 5′ caps and generally do not contain 3′ poly-adenylated tails; and translation is inhibited in chloroplasts and in bacteria by selective agents such as chloramphenicol.

Several RNA elements that act as mediators of translational regulation have been identified within the 5′UTR's of chloroplast mRNAs. These elements may interact with nuclear-encoded factors and generally do not resemble known prokaryotic regulatory sequences.

A vector or other recombinant nucleic acid molecule of the invention can include a nucleotide sequence encoding a reporter polypeptide or other selectable marker. The term “reporter” or selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation. A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain prokaryotic cells or plant cells or both that express the marker and, therefore, can be useful as a component of a vector of the invention. Examples of selectable markers include those that confer antimetabolite resistance, for example, dihydrofolate reductase, which confers resistance to methotrexate; neomycin phosphotransferase, which confers resistance to the aminoglycosides neomycin, kanamycin and paromycin; hygro, which confers resistance to hygromycin; trpB, which allows cells to utilize indole in place of tryptophan; hisD, which allows cells to utilize histinol in place of histidine; mannose-6-phosphate isomerase which allows cells to utilize mannose; ornithine decarboxylase, which confers resistance to the ornithine decarboxylase inhibitor, 2-(difluoromethyl)-DL-ornithine; and deaminase from Aspergillus terreus, which confers resistance to Blasticidin S. Additional selectable markers include those that confer herbicide resistance, for example, phosphinothricin acetyltransferase gene, which confers resistance to phosphinothricin, a mutant EPSP-synthase, which confers glyphosate resistance, a mutant acetolactate synthase, which confers imidazolione or sulfonylurea resistance, a mutant psbA, which confers resistance to atrazine, or a mutant protoporphyrinogen oxidase, or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants. Since a composition or a method of the invention can result in expression of a polypeptide in chloroplasts, it can be useful if a polypeptide conferring a selective advantage to a plant cell is operatively linked to a nucleotide sequence encoding a cellular localization motif such that the polypeptide is translocated to the cytosol, nucleus, or other subcellular organelle where, for example, a toxic effect due to the selectable marker is manifest.

The ability to passage a shuttle vector of the invention in a prokaryote allows for conveniently manipulating the vector. For example, a reaction mixture containing the vector and putative inserted polynucleotides of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. The shuttle vector then can be introduced into plant cell chloroplasts, wherein a polypeptide of interest can be expressed and, if desired, isolated according to a method of the invention.

A polynucleotide or recombinant nucleic acid molecule of the invention, which can be contained in a vector, including a vector of the invention, can be introduced into plant chloroplasts using any method known in the art. As used herein, the term “introducing” means transferring a polynucleotide into a cell, including a prokaryote or a plant cell, particularly a plant cell plastid. A polynucleotide can be introduced into a cell by a variety of methods, which are well known in the art and selected, in part, based on the particular host cell. For example, the polynucleotide can be introduced into a plant cell using a direct gene transfer method such as electroporation or microprojectile mediated (biolistic) transformation using a particle gun, or the “glass bead method”, vortexing in the presence of DNA-coated microfibers or by liposome-mediated transformation, transformation using wounded or enzyme-degraded immature embryos.

Plastid transformation is a routine and well known method for introducing a polynucleotide into a plant cell chloroplast. Chloroplast transformation involves introducing regions of chloroplast DNA flanking a desired nucleotide sequence into a suitable target tissue; using, for example, a biolistic or protoplast transformation method (e.g., calcium chloride or PEG mediated transformation). Fifty bp to 3 kb flanking nucleotide sequences of chloroplast genomic DNA allow homologous recombination of the vector with the chloroplast genome, and allow the replacement or modification of specific regions of the plastome. Using this method, point mutations in the chloroplast 16S rRNA and rps12 genes, which confer resistance to spectinomycin or streptomycin, can be utilized as selectable markers for transformation, and can result in stable homoplasmic transformants, at a frequency of approximately one per 100 bombardments of target tissues. The presence of cloning sites between these markers provides a convenient nucleotide sequence for making a chloroplast vector, including a vector of the invention. Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase. Approximately 15 to 20 cell division cycles following transformation are generally required to reach a homoplastidic state. Plastid expression, in which genes are inserted by homologous recombination into all of the up to several thousand copies of the circular plastid genome present in each plant cell, takes advantage of the enormous copy number advantage over nuclear-expressed genes to permit expression levels that can readily exceed 10% of the total soluble plant protein.

Known direct gene transfer methods, such as electroporation, also can be used to introduce a polynucleotide of the invention into a plant protoplast. Electrical impulses of high field strength reversibly permeabilize membranes allowing the introduction of the polynucleotide. Known methods of microinjection may also be performed. A transformed plant cell containing the introduced polynucleotide can be identified by detecting a phenotype due to the introduced polynucleotide, for example, expression of a reporter gene or a selectable marker.

Microprojectile mediated transformation also can be used to introduce a polynucleotide into a plant cell chloroplast. This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000™ particle gun (BioRad; Hercules Calif.). Methods for the transformation using biolistic methods are well known. Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery. The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, the glass bead agitation method, and the like.

Reporter genes have been successfully used in chloroplasts of higher plants, and high levels of recombinant protein expression have been reported. In addition, reporter genes have been used in the chloroplast of C. reinhardtii, but, in most cases very low amounts of protein were produced. Reporter genes greatly enhance the ability to monitor gene expression in a number of biological organisms. In chloroplasts of higher plants, beta-glucuronidase (uidA), neomycin phosphotransferase (nptII), adenosyl-3-adenyltransf-erase (aadA), and the Aequorea victoria GFP have been used as reporter genes. Each of these genes has attributes that make them useful reporters of chloroplast gene expression, such as ease of analysis, sensitivity, or the ability to examine expression in situ.

Effective concentrations of the compositions provided herein or pharmaceutically acceptable salts or other derivatives thereof are mixed with a suitable pharmaceutical carrier or vehicle. Derivatives of the compounds, such as salts of the compounds or prodrugs of the compounds may also be used in formulating effective pharmaceutical compositions. The concentrations of the compounds are effective for delivery of an amount, upon administration, that ameliorates the symptoms of the disease. Typically, the compositions are formulated for single dosage administration.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients.

The active compounds can be administered by any appropriate route, for example, orally, parenterally, intravenously, intradermally, subcutaneously, or topically, in liquid, semi-liquid or solid form and are formulated in a manner suitable for each route of administration. Preferred modes of administration include oral and parenteral modes of administration. The active compound is included in the pharmaceutically acceptable carrier in an amount sufficient to exert a therapeutically useful effect in the absence of undesirable side effects on the patient treated. In one aspect, treated may be performed by contacting cells with the fusion protein of the invention ex vivo.

The therapeutically effective concentration may be determined empirically by testing the compounds in known in vitro and in vivo systems as described herein or known to those of skill in this art and then extrapolated therefrom for dosages for humans.

The concentration of active compound in the drug composition will depend on absorption, inactivation and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, a fusion protein as set forth in SEQ ID NO:5, SEQ ID NO:7, SEQ ID NO:11 and SEQ ID NO:13 is disclosed, including fusion protein-containing compositions admixed with pharmaceutically acceptable carriers. In one aspect, such fusion protein compositions can be used to treat a subject with a proliferative cell disorder, including B-cell derived proliferative disorders. In another aspect, such a proliferative disorder includes, but is not limited to, neoplasias, such as B-cell lymphomas.

In one aspect, a composition may include the fusion protein in combination with chemotherapeutic compounds, where such a combination may be used to treat a subject in need thereof. In one aspect, such chemotherapeutics include, but are not limited to, Aclacinomycins, Actinomycins, Adriamycins, Ancitabines, Anthramycins, Azacitidines, Azaserines, 6-Azauridines, Bisantrenes, Bleomycins, Cactinomycins, Carmofurs, Carmustines, Carubicins, Carzinophilins, Chromomycins, Cisplatins, Cladribines, Cytarabines, Dactinomycins, Daunorubicins, Denopterins, 6-Diazo-5-Oxo-L-Norleucines, Doxifluridines, Doxorubicins, Edatrexates, Emitefurs, Enocitabines, Fepirubicins, Fludarabines, Fluorouracils, Gemcitabines, Idarubicins, Loxuridines, Menogarils, 6-Mercaptopurines, Methotrexates, Mithramycins, Mitomycins, Mycophenolic Acids, Nogalamycins, Olivomycines, Peplomycins, Pirarubicins, Piritrexims, Plicamycins, Porfiromycins, Pteropterins, Puromycins, Retinoic Acids, Streptonigrins, Streptozocins, Tagafurs, Tamoxifens, Thiamiprines, Thioguanines, Triamcinolones, Trimetrexates, Tubercidins, Vinblastines, Vincristines, Zinostatins, and Zorubicins.

The tablets, pills, capsules, troches and the like can contain any of the following ingredients, or compounds of a similar nature: a binder, such as microcrystalline cellulose, gum tragacanth and gelatin; an excipient such as starch and lactose, a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, and fruit flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings and flavors. The active materials can also be mixed with other active materials which do not impair the desired action, or with materials that supplement the desired action.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE I

### Experimental Protocols and Methods for Generation of Antibody-Toxin Fusions

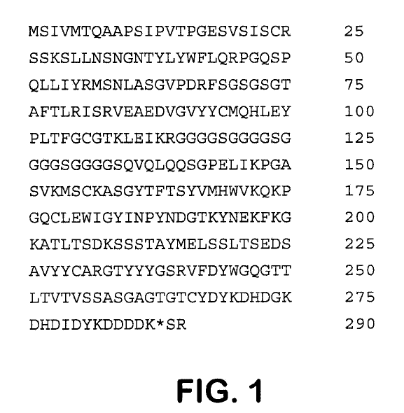

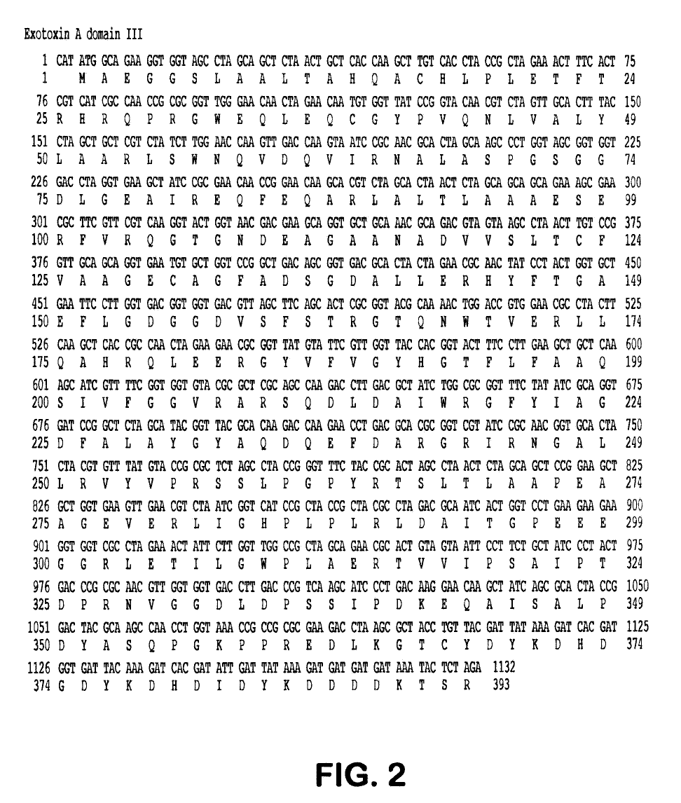

Synthesis of antibody and toxin genes, and construction of antibody-toxin fusion proteins. Coding regions for all recombinant proteins were synthesized de novo in C. reinhardtii chloroplast condon bias (Franklin et al. Plant J(2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). The coding regions synthesized include anti-human CD19 scFv (FIG. 1) antibody fragment (Meeker et al., Hybridoma (1984) 3:305-320), and domains II and III (FIG. 2) of Pseudomonas aeruginosa exotoxin A (Li et al., Proc Natl Acad Sci USA (1995) 92:9308-9312). The 5′ and 3′ terminal primers used in these assemblies contained restriction sites for Nde I, Xba I, respectively, for ease in subsequent cloning. A FLAG epitope tag was placed at the carboxy terminus of each protein, for analysis of protein expression and for subsequent purification using anti-flag affinity resin (Sigma, St. Louis, Mo.).

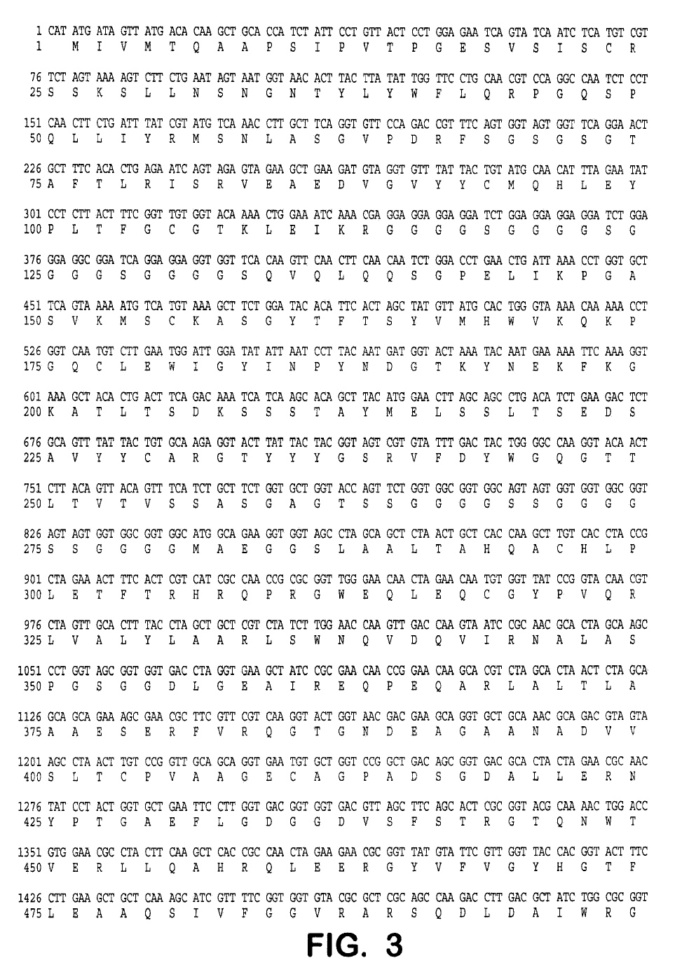

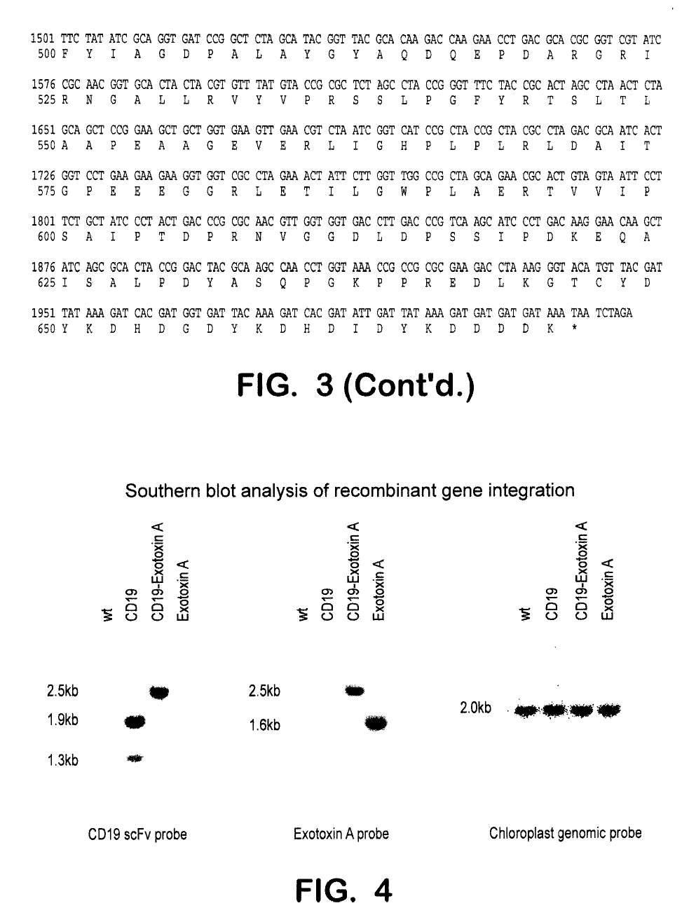

A CD19-ETA antibody-toxin fusion protein was generated by linking the CD19 scFv to ETA domains II and III using an inframe serine glycine amino acid linker (low complexity) located between the carboxy terminus of the antibody fragment and amino terminus of the toxin (FIG. 3). This fusion created a 2022-bp gene expressing a single polypeptide of 662 amino acids.

C. Reinhardtii transformation and growth conditions. For expression of the CD19 scFv, the atpA promoter and 5′ UTR and the rbcL 3′ UTR were used. For expression of ETA and CD19-ETA fusion the psbA promoter and 5′ UTR, and psbA 3′ UTR were used. Each of these promoters and UTRs were generated as previously described (Barnes et al., Mol Genet Genomics (2005) 274:625-636). The CD19 scFv expression cassette was placed in the Bam-HI site of integration plasmid p322 (Franklin et al., 2002), while the ETA and CD19-ETA expression cassettes contained flanking genomic sequences of the psbA gene that allowed for homologous recombination into the C. reinhardtii chloroplast genome as a replacement of the endogenous psbA gene (Manuell et al., Plant Biotech J (2007)).

C. reinhardtii strain 137c was grown in TAP medium (Gorman and Levine, Proc Natl Acad Sci USA (1965) 54:1665-1669) containing 1 mM 5-Fluorodeoxyuridine (FUDR) to late log phase under illumination of 4000 lux. Cells were pelleted by centrifugation and resuspended in TAP medium and 0.5×10⁸ cells were plated on agar plates containing TAP medium with 150 mg/L spectinomycin. The ETA, CD19, and CD19-ETA expression cassettes were transformed separately into 137c cells along with the spectinomycin resistance ribosomal gene of plasmid p228 (Chlamydomonas Stock Center, Duke University). Colonies that grew on spectinomycin plates were screened by Southern blot for the presence of the CD19 scFv or ETA sequences, and transformants positive for the correct gene were taken through additional rounds of selection on specinomycin plates in order to obtain transformants that were homoplastic for each gene.

Southern and Northern blots. Southern blots and ³²P labeling of DNA for use as probes were carried out as described in Sambrook et al. (Molecular Cloning: A laboratory Manual, (1989), Cold Spring Harbor Laboratory Press, Cold Spring Harbor, N.Y.), and Cohen et al. (Methods Enzymol (1998) 297:192-208). Genomic DNA from wt and the three transgenic lines was digested with restriction endonucleases, separated on agarose gels, and blotted to nylon membrane prior to being hybridized with ³²P-labeled probes. The probes for Southern and Northern blots included a 2.0 kb BamHI/XhoI fragment from the 3′ end of the psbA locus, a 1182-bp coding region from ETA, a 674-bp coding region from CD19 scFv and a 600 bp fragment of the psbA cDNA. Northern and Southern blots were visualized utilizing a Packard Cyclone Storage Phosphor System™ equipped with Optiquant™ software.

Recombinant protein expression and characterization. C. reinhardtii proteins were isolated using a lysis buffer containing 10 mM Tris, 600 mM NaCl, 15% sucrose and 1 mM PMSF. To each gram of cell pellet 10 ml of lysis buffer was added and the cell ruptured by sonication. The insoluble and soluble phases were separated by centrifugation at 25,000×g. Protein concentration was determined by Lowry protein assays. Proteins were separated by SDS-PAGE and blotted to a nitrocellulose membrane. Individual proteins were identified using antisera for ETA (Sigma, St. Louis, Mo.), or mouse anti-M2 Flag antibody (Sigma, St. Louis, Mo.). After washing with TBST the membranes were decorated with either a goat anti-rabbit antibody for ETA (Southern Biotech, Birmingham, Ala.), or a goat anti-mouse antibody for anti-flag (Southern Biotech, Birmingham, Ala.), both secondary antibodies were conjugated with alkaline phosphatase. The decorated proteins were visualized using alkaline phosphate assay.

For purification of the proteins, crude soluble extracts were incubated with M2 Flag resin following the method as described (Sigma, St. Louis, Mo.). The immuno-affinity purified proteins were eluted from the matrix with 100 mM Glycine and 600 mM NaCl pH 3.5 and then dialyzed against phosphate buffered saline (137 mM NaCl, 2.7 mM KCl, 1.8 mM K₂HPO₄, 10 mM Na₂HPO₄, pH 7.4). Purified proteins were used for bio-activity assays as described below.

In vitro toxin activity assays. To test for bio-activity of the exotoxin A protein, an in vitro ADP-ribosyltransferase assay was performed. The bio-activity of exotoxin A results from the catalytic transfer of ADP-ribose from NAD⁺ to eukaryotic elongation factor 2 (eEF2). The reaction mixture contained 20 mM tris pH 8.2, 50 μg/ml BSA, 1 mM EDTA, 1 mM DTT, 1 ng/μl eEF2 (wheat germ, Karen Browning UT, Austin), and 1.2 μl NAD⁺ mixture (1 μl ³²P NAD⁺ 800 ci/mmol added to 28.4 μl of NAD⁺ 40 mg/ml stock and the volume brought up to 800 μl with water) per 10 μl reaction. Fifty ng of purified ETA or purified CD19-ETA was added to each reaction. As negative controls, 50 ng of purified CD19 alone, or 50 ng of crude C. reinhardtii soluble proteins, were used. The reaction mix was incubated 10 minutes at room temperature before a 1× volume of protein loading dye was added and the proteins separated by SDS-PAGE. Following separation, the gel was dried and placed on a phosphorImager screen and viewed using a Packard Cyclone phosphorImager System™ using Optiquant™ software.

Isolation of peripheral blood lymphocytes from whole blood of normal donors. Initially, 15 mls of whole blood was mixed with 15 mls of phosphate buffered saline (PBS) and underplayed with 10 ml of Ficoll Hypaque in a Falcon conical tube before centrifugation at 1750 rpm for 25 min. After centrifugation the layer containing the PBLs was removed and washed twice with PBS. A cell count was performed and the percentage of B cells in the PBL mixture was determined by FACS flow cytometry using CD20 or CD19 antibody hybridization.

Activation of B cells in PBL. Cells were resuspended in RPMI culture media with HEPES and PSG and 5×10⁵−1×10⁶ cells were added to each well of a 96 well plate. Twenty μl of anti-CD40 antibody at a concentration of 1 μg/ml was added to each well. The plate was then incubated at 4° C. for 1-2 hours. Following the incubation at 4° C., an IgG anti-STAR81 was added to each well to final concentration of 0.2 μg/ml. The plate was then incubated at 37° C. for an additional 1-2 hours. After the final incubation, 20 μl of IL2 and IL10 were added from a stock at 100 ng/ml to complete the activation of PBLs.

CD19 antibody binding to Ramos and PBL cells. 1×10⁶ cultured Ramos B cells or activated PBL were contacted with increasing amounts of CD19-ETA for one hour in FACS buffer (1×PBS, 2% FCS and 0.05% sodium azide) at 4° C. CD19-ETA concentrations from 0.2 μg to 2.5 μg were used for binding assays and from 0.2 μg to 7.5 μg were used for cell killing assays. As controls, a commercially supplied anti-CD19 antibody conjugated with PE (Southern Biotech, Birmingham, Ala.) was used to verify that the target antigen, CD19, was located on the cell surface of both cell types. B cell line Molt 4, which is CD19 negative, was used as a negative control to confirm that the CD19-ETA fusion protein was not binding nonspecifically to the B cells. After 1 hour incubation with CD 19-ETA, the cells were washed three times with FACS buffer and then incubated with an anti-Flag antibody conjugated with FITC for 45 mins. After the incubation, cells were washed an additional three times and resuspended in 500 μl of FACS buffer and analyzed by FACS flow cytometry.

Ramos and PBL cell killing assay. Both Ramos cells and activated PBL were added to a 96 well plate at 1×10⁶ cells/well and cultured for 24 hours. The cells are then treated with varying amounts of CD19-ETA. As negative controls, the cells were also treated with CD 19 scFv or with exotoxin A, both expressed and purified from C. reinhardtii. Following 24 hours of incubation the cells were stained with annexin-5 antibody conjugated with FITC, or with propidium iodine (PI) and analyzed by FACS flow cytometry. Cell killing associated with apoptosis results in increased annexin-5 staining.

### EXAMPLE II

### Synthesis and Assembly of an Antibody-Toxin Fusion and Construction of Chloroplast Expression Vectors

In order to obtain high levels of protein expression in algal chloroplasts, transgene codons need to be optimized to reflect abundantly expressed genes of the C. reinhardtii chloroplast (Franklin et al., 2002; Mayfield et al., 2003; Mayfield and Schultz, 2004). Two recombinant protein codon regions were designed, a single chain antibody fragment that binds to CD19 protein found on human B cells (Meeker et al., 1984), and a truncated exotoxin A protein from Pseudomonas aeruginosa (Li et al., 1995) that lacks the cell binding domain, but retains the translocation and catalytic domains of the toxin. The amino acid sequences of the original proteins were maintained, but the codon usage was changed to reflect that of highly expressed C. reinhardtii chloroplast genes. The resulting chloroplast-optimized CD19 scFv coding sequence (CD19, FIG. 1) was cloned into an expression cassette that contained the atpA promoter and 5′ UTR and the rbcL 3′ UTR, in the p322 expression cassette (Franklin et al., 2002). This cassette allows for transgene integration by homologous recombination between the psbA gene and the 16S rRNA gene in the inverted repeat of the chloroplast genome. The truncated exotoxin A protein (domains II and III) coding region (ETA, FIG. 2) was cloned downstream of the psbA promoter and 5′ UTR and upstream of the psbA 3′ UTR (Manuell et al., 2007). The genomic sequences flanking the psbA 5′ and 3′ UTRs were also included to facilitate homologous replacement of the endogenous psbA gene (Manuell et al., 2007).

For assembly of the antibody toxin fusion, an inframe Kpn I restriction site was placed at the carboxy end of the CD19 scFv, and a corresponding inframe Kpn I site, along with a flexible amino acid linker, was placed at the amino terminus of the exotoxin A domain II gene. Ligation of these two fragments resulted in a fusion protein containing the CD19 scFv as the amino half of the protein and exotoxin A domain II and III as the carboxy half of the protein (CD19-ETA, FIG. 3). The Kpn I site was subsequently removed by site directed mutagenesis. The CD19-ETA gene was ligated into the same psbA vector as the ETA gene to allow for integration into the chloroplast genome as a replacement of the psbA gene.

### EXAMPLE III

### Introduction of the Recombinant Genes into the C. Reinhardtii Chloroplast Genome

The chimeric CD19, ETA, and CD19-ETA genes were introduced into the C. reinhardtii chloroplast genome by particle bombardment along with a selectable marker gene conferring spectinomycin resistance (Franklin et al., 2002). Spectinomycin resistant transformants were screened for the presence of the transgenes by Southern blot analysis. Chloroplasts contain multiple copies of their genome and several rounds of selection are required to achieve a homoplasmic strain with all copies of the organelle genome uniformly transformed. Using probes to both the coding regions of CD19, ETA, or a flanking genome region, Southern blot analysis identified homoplastic lines for each of the three recombinant proteins (FIG. 4). Hybridization of the blots with an ETA coding region probe identified a 1.6 kb band in ETA strain 1-4 and a 2.5 kb band in CD19-ETA strain 2-11, while hybridizing with a CD19 coding region probe identified a 2.5 kb band in the CD19-ETA strain 2-11 and a 1.3 kb band in the CD19 strain. Neither the CD19 or ETA genes were detected in the wt strain. Hybridization with a probe from the 3′ end of the psbA locus yielded the expected 2.0 kb band in all samples.

### EXAMPLE IV

### Accumulation of Recombinant mRNAs in Transgenic Strains

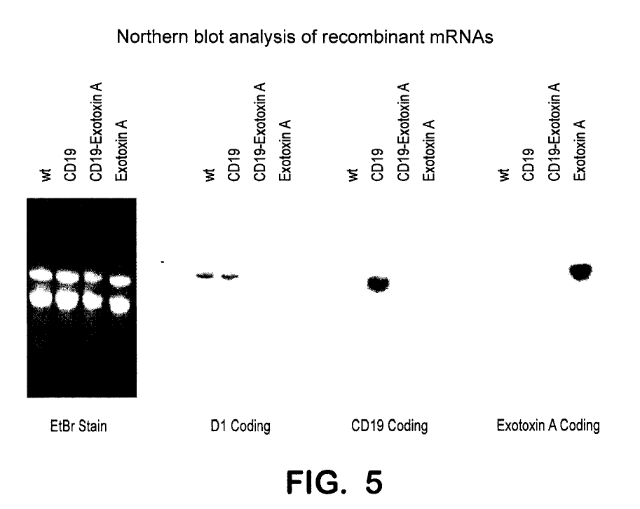

Northern blot analysis of total RNA was used to determine if the recombinant genes were transcribed correctly in transgenic C. reinhardtii chloroplasts. Ten μg of total RNA, isolated from wt and the three transgenic lines, was separated on denaturing agarose gels and blotted to nylon membrane. Duplicate filters were stained with ethidium bromide (FIG. 5, left panel), or hybridized with a ³²P labeled psbA cDNA (FIG. 5, central panel) a CD19 coding region probe (FIG. 5, central panel), or an ETA coding region probe (FIG. 5, right panel). Each of the strains accumulated equal amounts of total RNA (stained bands), demonstrating that equal amounts of RNA were loaded for each lane, and that chloroplast transcription and mRNA accumulation are normal in the transgenic lines. The ETA probe identified an mRNA of approximately 2.2 kb in the ETA transgenic lane, and 3.1 kb in the CD19-ETA lane, while CD19 probe identified the same 3.1 kb mRNA in the CD19-ETA lane and a 2.0 kb mRNA in the CD19 lane. A psbA cDNA probe recognized the 1.4 kb psbA mRNA in both the wt and CD19 strains, but not in the ETA or CD19-ETA lanes, confirming that both ETA and CD19-ETA integration resulted in complete deletion of the endogenous psbA gene (Manuell et al., 2007).

### EXAMPLE V

### Analysis of CD19, ETA, and CD19-ETA Protein Accumulation in Transgenic C. Reinhardtii Chloroplasts

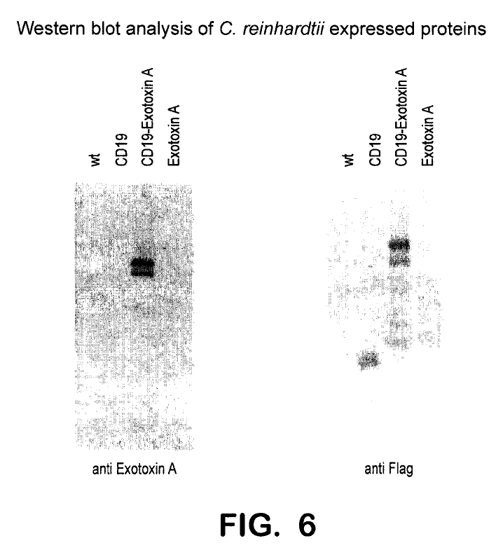

Protein accumulation in transgenic lines was monitored by Western blot analysis. Twenty μg of total soluble protein (tsp) from wt and the transgenic lines was separated by SDS-PAGE and blotted to nitrocellulose membrane. Blots were hybridized with either an anti-ETA antibody (FIG. 6, left panel) or anti-flag antibody (FIG. 6, right panel). The anti-ETA antisera recognized a protein of 42 kDa in the ETA transgenic line and a protein of 71 kDa in the CD19-ETA in the transgenic line. The anti-Flag antisera recognized the same two proteins, as well as the 30 kDa CD19 protein. Additional bands (likely degradation products) were detectable with anti-Flag in the CD 19-ETA and ETA lanes.

### EXAMPLE VI

### Bioactivity of C. Reinhardtii Chloroplast Expressed Exotoxin a Protein In Vitro

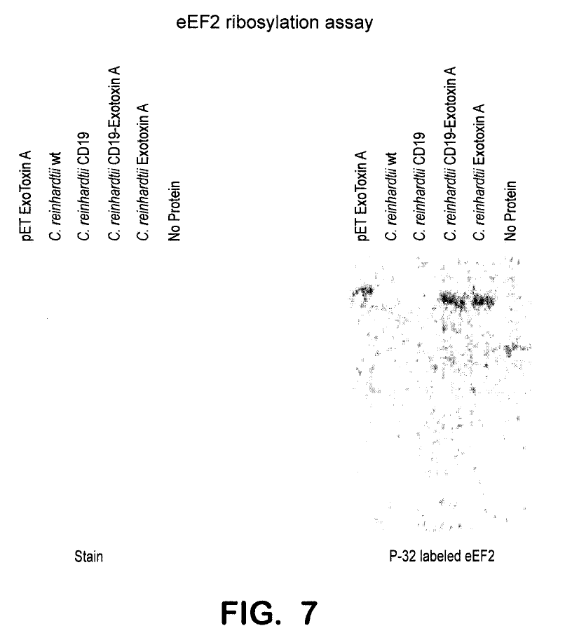

In vitro ADP-ribosyltransferase assays were performed to detect exotoxin A-specific ribosylation of elongation factor 2 (eEF2) using purified eEF2 from wheat germ and radio-labeled NAD⁺. As shown in FIG. 7, the 93 kDa eEF2 is labeled with ADP from NAD⁺ when treated with purified ETA protein expressed in E. coli (lane 1), and when treated with chloroplast expressed and purified ETA protein expressed and purified ETA (lane 3) or CD19-ETA (lane 4). No labeled eEF2 was observed in controls lacking exotoxin A.

### EXAMPLE VII

### CD19 Binding and Cell Killing Ability of the CD19-ETA Fusion Protein

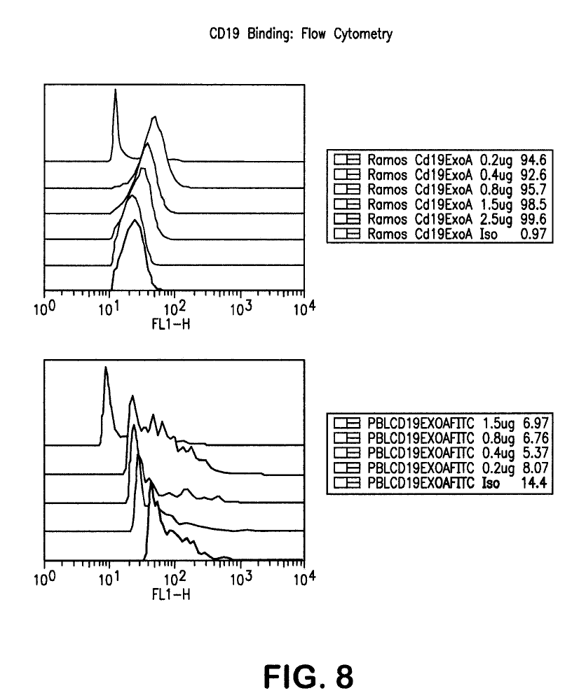

CD19-ETA binding to CD19-positive human cells was measured using flow cytometry and a fluorescently labeled secondary antibody directed against the Flag epitope found on the carboxy end of the CD19-ETA fusion protein. The human immortalized Ramos B-cell line, and activated human peripheral blood lymphocytes (PBLs) both express CD19. Increasing concentrations of CD19-ETA were added to both Ramos and PBL cells followed by the addition of FITC labeled anti-Flag antibodies, after which the cells were analyzed by flow cytometry. As shown in FIG. 8, a concentration-dependent shift in fluorescence was observed in both cell types, demonstrating that B-cells were bound by the CD19-ETA in proportion to the amount of fusion protein added.

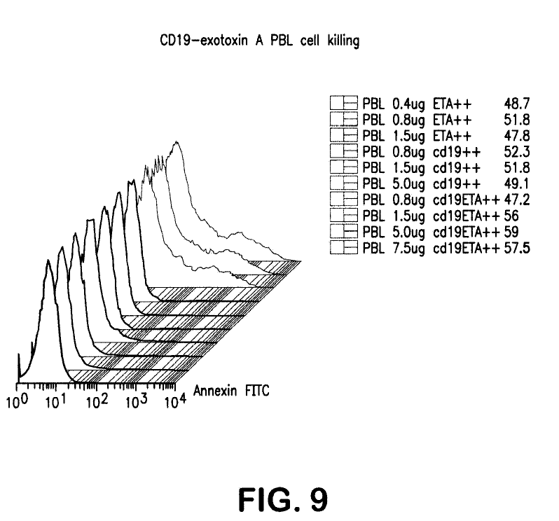

To determine if the CD19-ETA bound to the cells was endocytosed and killed the cells, apoptosis was measured using annexin A5 staining. Annexin A5 detects phosphatidylserine on the cell surface, a marker associated with programmed cell death (Koopman et al., 1994). Conjugation of annexin A5 with FITC thus reveals cell killing by increased fluorescence of cells expressing the annexin A5 ligand. As shown in FIG. 9, treatment of PBLs with the CD19 scFv alone had no effect on fluorescence even after a 24 hour incubation. Treatment with exotoxin A domain alone also failed to induce cell killing. However, treatment of PBLs with increasing amounts of CD19-ETA resulted in increased fluorescence, indicating that the CD19-ETA, but not CD19 or ETA alone, induces phosphatidylserine suggestive of concentration-dependent cell killing.

### EXAMPLE VIII

### Production of a Ribosome Inactivating Protein, Gelonin, in Algal Chloroplasts

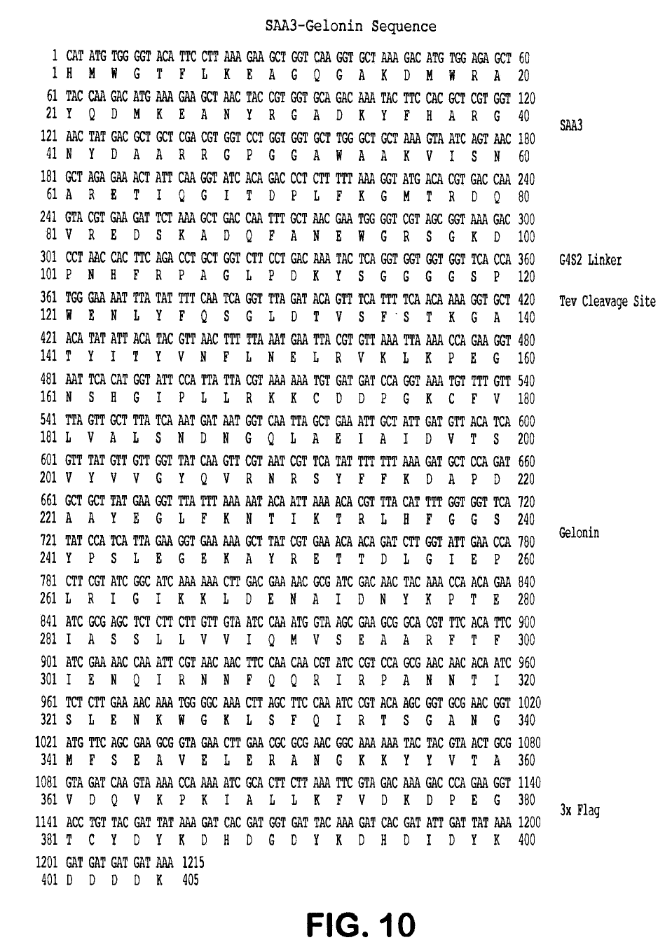

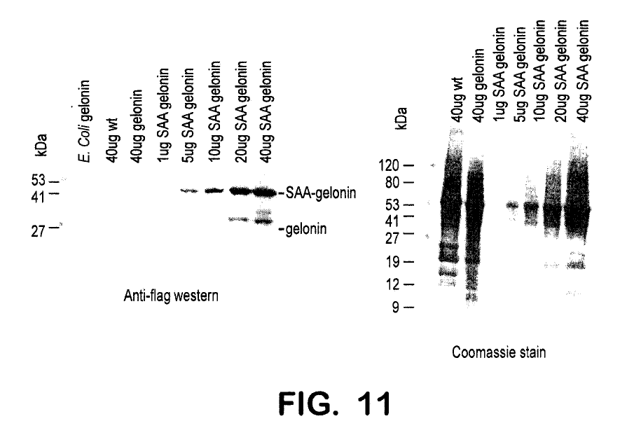

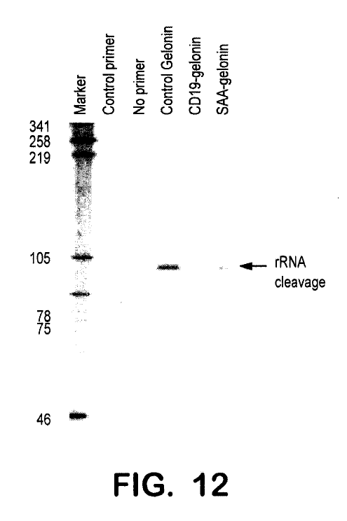

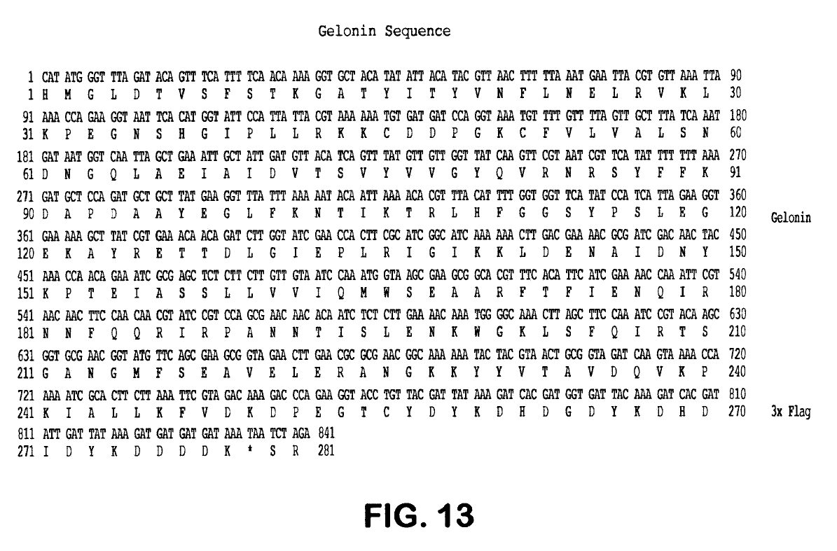

To determine if eukaryotic toxins in addition to ETA could also be produced in algal chloroplasts a gene encoding a codon optimized ribosome inactivating protein, gelonin was generated. Not to be bound by theory, however, gelonin seems to inactivate 80S eukaryotic ribosomes resulting in cell death, but does not inactivate bacterial ribosomes or chloroplast ribosomes. An SAA-gelonin fusion protein was constructed for expression in algal chloroplasts. FIG. 10 shows the nucleotide and amino acids sequence of the SAA-nGelonin fusion protein (SEQ ID NOS:6 and 7). Amino acid residues 1 to 113 define the codon optimized bovine serum amyloid A 3 protein, amino acid residues 114 to 119 define the flexible peptide linker, amino acid residues 120 to 128 define a TEV protease site, amino acid residues 129 to 379 define native Gelonin, and amino acid residues 380 to 405 at the carboxy terminus define the FLAG epitope tag. FIG. 11 shows a western blot analysis of recombinant rGelonin and SAA-nGelonin protein accumulation in C. reinhardtii transgenic chloroplasts. Total proteins from wt, a transgenic line expressing rGel, and a dilution series of proteins from a transgenic line expressing SAA-nGelonin are shown. The proteins were blotted to membranes and decorated with anti-FLAG (right panel) antisera. In vitro activity assay of isolated chloroplast expressed SAA-nGelonin is shown in FIG. 12. Lane 2 shows a control primer extension product. Lane 3 shows primer extension with no added protein, lane 4 shows primer extension with bacterially expressed rGelonin added, and lane 6 shows primer extension with purified SAA-nGelonin added. These data demonstrate that eukaryotic 80S ribosome inactivating proteins can be expressed in algal chloroplasts, and that chloroplast are capable of expressing a variety of eukaryotic toxins.

### EXAMPLE IX

### Fc-ETA Fusion Protein

The amino acid sequence for ETA to be used will be as above. Briefly, a chloroplast biased nucleotide sequence is generated which encodes ETA (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ETA and amino terminus of the Fc region. The fusion protein may be purified by Protein A or Protein G affinity chromatography.

### EXAMPLE X

### Fc-Gelonin Fusion Protein

The amino acid sequence for gelonin to be used will be as above. Briefly, a chloroplast biased nucleotide sequence is generated which encodes gelonin (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the gelonin and amino terminus of the Fc region. Again, the fusion protein may be purified by Protein A or Protein G affinity chromatography.

### EXAMPLE XI

### In Vivo CD19-ETA Immunotoxin Fusion Activity

The bioactivity of CD19-ETA and other immunotoxin fusions with respect to clearance and cell killing is analyzed in an implanted human B cell lymphoma animal model. The Ramos cell line is a well-established model for human B cell lymphomas and has proven useful to provide a clear proof of concept that the algae-produced CD19-ETA toxin construct binds and efficiently kills the Ramos cells in vitro. Cell death occurs within 24 hours of exposure to the fusion protein. To establish the proof of killing activity in vivo a Ramos cell line engineered to constitutively express the firefly luciferase gene will be created. These luciferase-labeled Ramos cells (Ramos/luc) will be implanted in a single Matrigel™ scaffold in the abdominal wall of immunodeficient NOD/SCID mice to form a discrete tumor. Intraperitoneal injection of the luciferase substrate, luciferin, will result in a light emission from the labeled tumor cells that is imaged using a Xenogen instrument. The advantages of this approach is that imaging is done on anesthetized, live animals allowing the tumor's progression or destruction to be followed serially over time and as a function of CD19-ETA dose with a high degree of accuracy and sensitivity. With this technology, multiple animals including controls can be readily imaged in a single experiment. A second approach will be to implant the Ramos/luc by injection directly into the blood stream via tail vein injection. This results in a general dissemination of the lymphoma cells, particularly to spleen, lungs and liver, very much like a human clinical presentation of Stage III or IV lymphoma. The Xenogen luciferase imaging technology is also well-suited to detection and measurement of this type of multiple small tumor metastases. The objectives of these studies will be to demonstrate the capability of the CD19-ETA construct to kill both a discrete tumor and disseminated disease, and to establish the total required dose, time frame and correlations with achieved serum levels of the fusion protein to achieve these effects. Another critical question for these preclinical studies is the ability of the CD19-ETA construct to efficiently enter and kill lymphoma cells within discrete tissue compartments such as spleen and liver. As additional constructs are considered, the issue of how additional toxin candidates and increasingly larger and more complex proteins function in tissue compartments becomes critical, because increased in vitro binding or killing efficiency is not useful if the new constructs cannot readily penetrate to the local site of the tumor cell in vivo. Additional studies include injection of another B cell lymphoma line and a survey of implanting multiple naturally occurring B cell lymphoma cells derived from human patients. Studies will also involve testing the immune response to this therapeutic protein. The initial studies will be done in immunodeficient NOD/SCID mice that mount no immune response to either the implantation of the human tumor cells or the CD19-ETA. Thus, studies of immune responses to the fusion protein will be done in fully immunocompetent mouse strains such as C57/B16, C3Hej and Balb/c. It is expected that following administration of a therapeutically effective dose of anti-CD19-ETA immunotoxin a concentration dependent killing of human lymphoma cells and concomitant loss of luciferase luminescence is observed.

### EXAMPLE XII

### Full-Length Antibody-Toxin Fusions

Chloroplasts are eukaryotic organelles that contain a number of chaperones normally used for folding and assembly of complex photosynthetic proteins imported into chloroplast from the cytoplasm. Chloroplasts have also been shown to have protein disulfide isomerases, and plastids have been shown to be able to form correct disulfide bonds in recombinant human somatotropin, and to assemble correctly disulfide linked complex human antibodies, processes that bacterial are generally unable to complete. The ability to assemble complex human antibodies in an environment that allows for toxin synthesis and accumulation, should allow for the synthesis and assembly of full-length human antibody-toxin fusion proteins. Full length heavy chain protein genes, from antibodies directed against CD19, CD22, or any appropriate cell surface antigen, will be constructed with a restriction site on the carboxy end of the heavy chain coding region to allow for the inframe fusion of a toxin domain. The resulting heavy chain-toxin protein gene will be transformed in plastids, along with a corresponding light chain gene, so that both proteins will be synthesized within the same plastid. Simultaneous expression of light chain and heavy chain-toxin proteins in chloroplasts will allow for the assembly of a full length antibody containing a toxin domain on the carboxy end of the heavy chain protein. Expression in this way should allow for unobstructed binding to the appropriate antigen from the variable regions of the light and heavy chain proteins as well as increased stability of the antibody-toxin protein brought about by the stabilizing effects of the heavy chain constant domains. Similar constructs will be made using a Fab fragment of the heavy chain with an appropriate site on the carboxy end of the heavy chain protein to fuse an inframe toxin domain. Co-expression of a Fab heavy chain-toxin protein with the appropriate light chain protein should result in a Fab-toxin fusion protein containing two antigen binding domains and two toxin domains, resulting in a potentially superior cell binding and killing molecule.

### EXAMPLE XIII

### CD19 scFv-Gelonin-Toxin Fusions

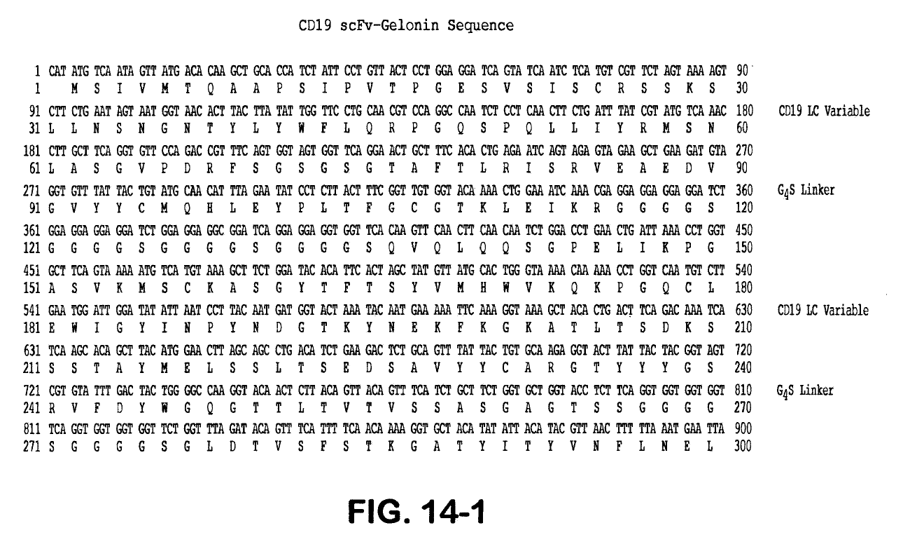

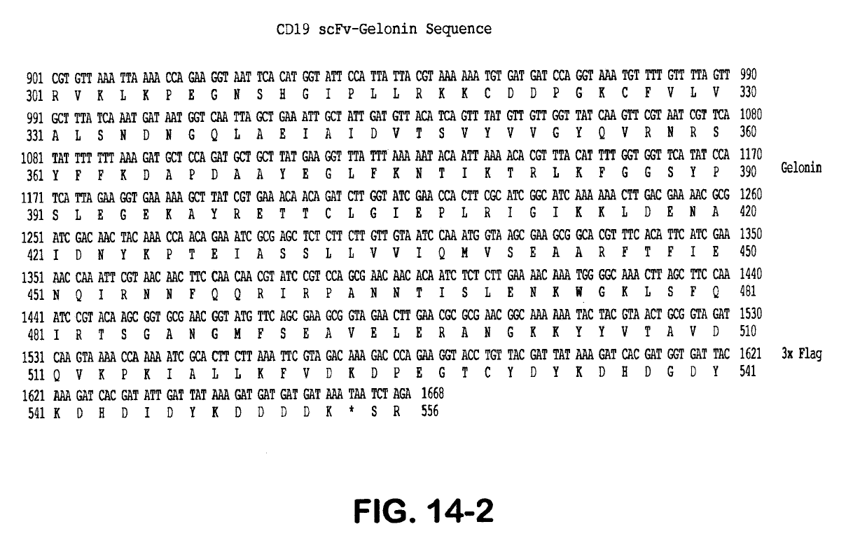

A CD19 scFv-Gelonin fusion protein was generated as shown in FIG. 14 as described herein (SEQ ID NOS:10 and 11, respectively). Amino acid residues 1 to 115 define the variable regions of the light chain, amino acid residues 116 to 135 define the flexible peptide linker, amino acid residues 136 to 264 define the variable region of the heavy chain, amino acid residues 265 to 276 define the flexible peptide linker, amino acid residues 277 to 527 define native Gelonin, and amino acid residues 528 to 556 at the carboxy terminus define the FLAG epitope tag.

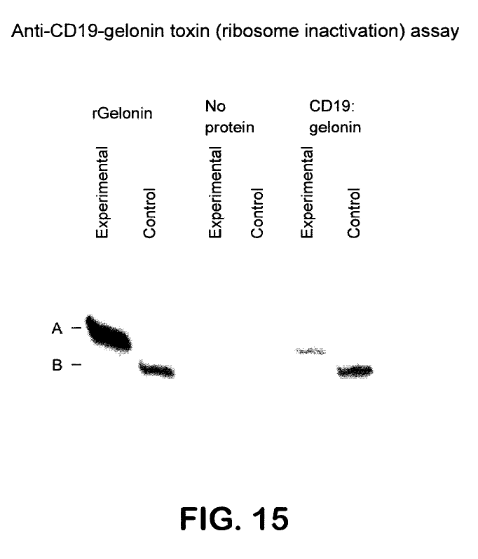

An in vitro gelonin assay was performed using the algal expressed CD19 scFv-Gelonin fusion protein. Gelonin activity is assayed by primer extension with radio-labeled primer. Yeast ribosomes were treated with purified recombinant gelonin, CD19:Gelonin, or untreated (no protein). Active gelonin will cleave the rRNA within the ricin loop. After treatment rRNA is isolated and used as a template for primer extension. ‘Experimental’ primers will give a product if gelonin activity is present (FIG. 15A). ‘Control’ primers will give a product (FIG. 15B) if rRNA is present.

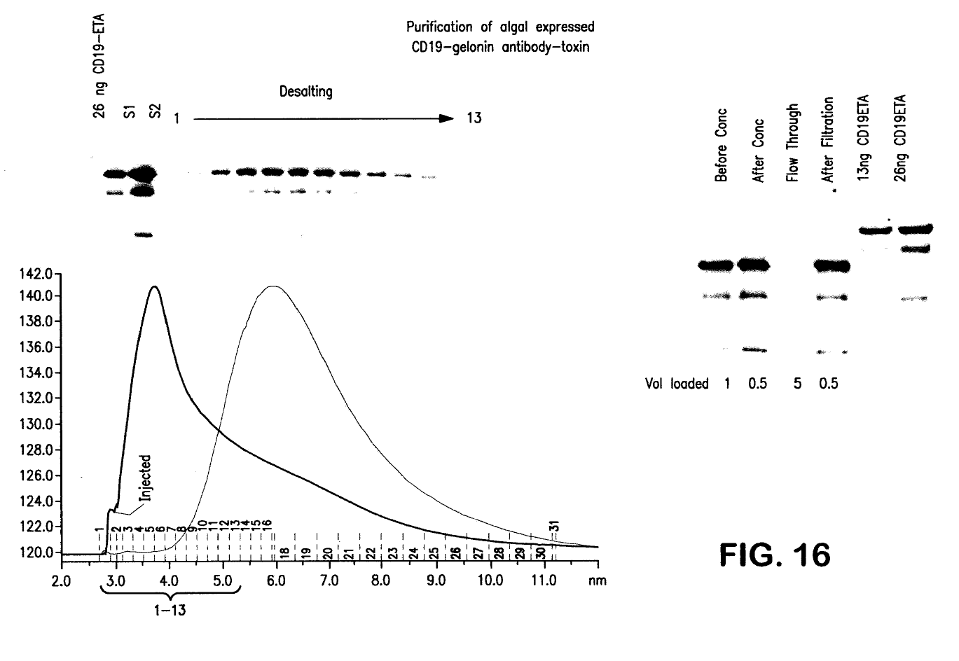

As shown in FIG. 16, the algal expressed CD19 scFv-Gelonin fusion protein was purified. FIG. 16A shows a Western blot of starting material, purified by FLAG affinity from crude algae lysate, before and after concentration (S1 and S2 respectively), then elutions from desalting column. FIG. 16B shows the elution profile from desalting column. Darker line shows UV absorbance, lighter line shows conductivity (salt). FIG. 16C shows a Western blot of purified desalted samples. Elutions 2-10 from desalting column were pooled (lane 1) and concentrated (lane 2), and filtered (lane 4).

### EXAMPLE XIV

### CD19 scFv-CH2-ETA-Toxin Fusions

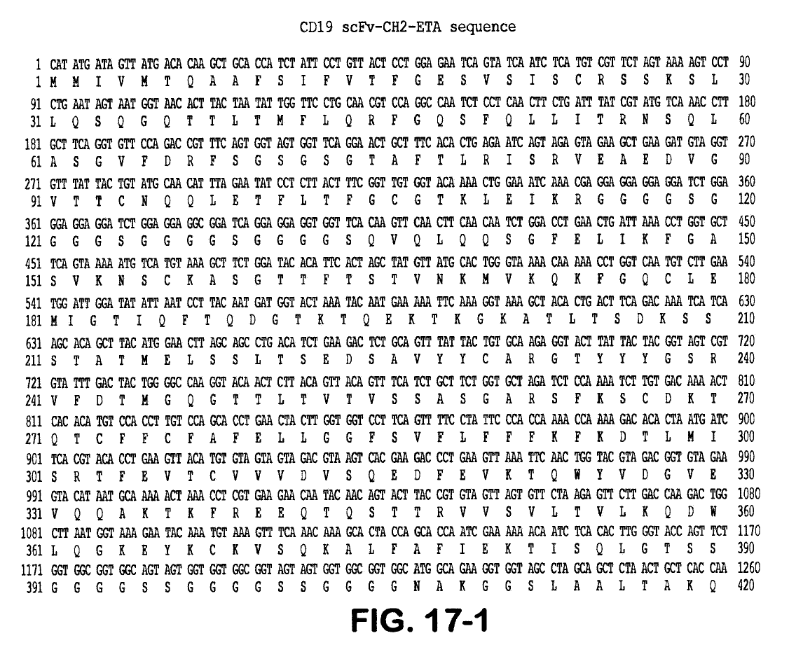

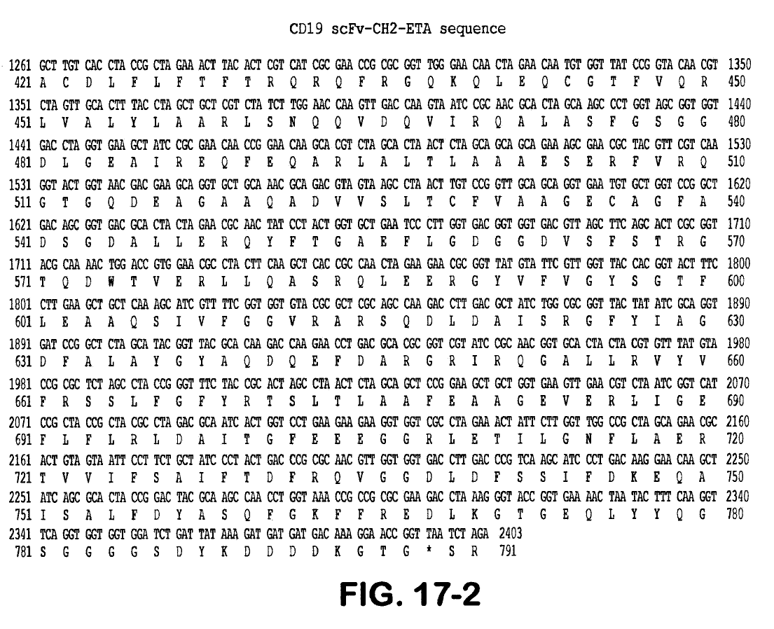

A CD19 scFv-CH2-ETA fusion protein was generated as shown in FIG. 17 as described herein (SEQ ID NOS:12 and 13, respectively). Amino acid residues 1 to 261 define the variable regions of the light chain, amino acid residues 262 to 381 define the CH2 constant domain, amino acid residues 382 to 772 define Exotoxin A, amino acid residues 773 to 780 define a TEV cleavage site, amino acid residues 781 to 786 define the flexible peptide linker, and amino acid residues 782 to 791 at the carboxy terminus define the FLAG epitope tag.

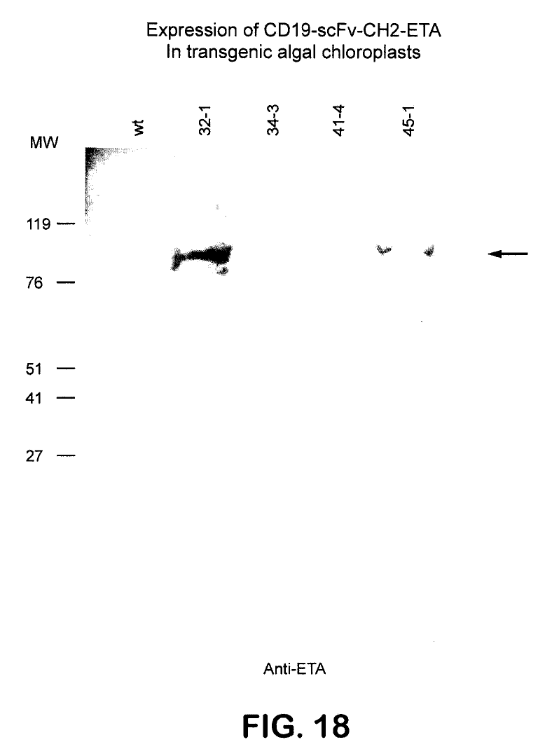

FIG. 18 shows algal expression of an anti-CD19-scFv-heavy chain CH2 domain-exotoxin A chimeric protein. Four transgenic lines, 32-1, 34-3, 41-4 and 45-1 were analyzed by western blot analysis for the accumulation of the chimeric protein. Protein from non-transformed wild type cells (Wt) was loaded in Lane 1. The chimeric antibody-toxin protein (arrowhead) accumulates as a soluble protein at the correct molecular weight (85 kD) in at least three of the transgenic lines, 32-1, 41-4 and 45-1. The chimeric protein was visualized using an anti-ETA antibody.

Although the invention has been described with reference to the above examples, it will be understood that modifications and variations are encompassed within the spirit and scope of the invention. Accordingly, the invention is limited only by the following claims.

What is claimed is:

1. A nucleic acid construct comprising in operable linkage:
a) nucleic acid signaling elements for homologous recombination and expression of the fusion protein in a plant or algae plastid; and
b) a first polynucleotide sequence encoding a first polypeptide and a second polynucleotide sequence encoding a toxin, wherein the first and second polynucleotide sequences are expressed as a fusion protein.

2. The construct of claim 1, wherein the first polynucleotide encodes a binding domain.

3. The construct of claim 1, wherein the binding domain comprises an antibody or an antigen binding fragment thereof.

4. The construct of claim 3, wherein the antibody is a complete antibody.

5. The construct of claim 3, wherein the binding domain consists essentially of an Fc region.

6. The construct of claim 5, wherein the Fc region is hIgG1Fc.

7. The construct of claim 2, wherein the binding domain recognizes a cell surface marker.

8. The construct of claim 7, wherein the cell surface marker is preferentially expressed on B-cells.

9. The construct of claim 7, wherein the cell surface marker is CD19.

10. The construct of claim 1, wherein the first polynucleotide encodes mammary associated serum amyloid (SAA).

11. The construct of claim 1, wherein the toxin is functional in a eukaryotic cell.

12. The construct of claim 1, wherein the toxin is an endotoxin or exotoxin.

13. The construct of claim 12, wherein the toxin is exotoxin A.

14. The construct of claim 10, wherein the toxin is obtained from a plant.

15. The construct of claim 14, wherein the plant toxin is gelonin.

16. A plant cell or algae cell or progeny thereof comprising the construct of claim 1.

17. A plant cell or algae cell plastid comprising the construct of claim 1.

18. The plant cell, algae cell or progeny of claim 16, wherein the first and second polynucleotides are stably integrated into the plastid of the cell.

19. A vector comprising the construct of claim 1.

20. A method of producing a bifunctional fusion protein comprising:
i) contacting a plastid with one or more expression constructs, wherein the expression constructs comprise, in operably linkage: a) a nucleic acid signal element for homologous recombination and expression of the fusion protein in the plastid; and b) a first polynucleotide sequence encoding a first polypeptide and a second polynucleotide sequence encoding a toxin, wherein the first and second polynucleotide sequences are expressed as a fusion protein;
ii) allowing the construct to integrate into the genome of the plastid; and
iii) expressing the fusion protein encoded by the construct.

21. The method of claim 20, wherein the plastid is in a plant cell or algae cell or progeny thereof.

22. The method of claim 20, wherein the first polynucleotide encodes an antibody or an antigen binding fragment thereof.

23. The method of claim 20, wherein the first polynucleotide encodes a fragment consisting of an Fc region.

24. The method of claim 23, wherein the Fc region is hIgG1Fc.

25. The method of claim 22, wherein the binding domain recognizes a cell surface marker.

26. The method of claim 22, wherein the binding domain recognizes a cell surface marker expressed on B-cells.

27. The method of claim 26, wherein the cell surface marker is CD19.

28. The method of claim 20, wherein the first polynucleotide encodes mammary associated serum amyloid (SAA).

29. The method of claim 20, wherein the toxin is an endotoxin or exotoxin.

30. The method of claim 29, wherein the toxin is exotoxin A.

31. The method of claim 28, wherein the toxin is obtained from a plant.

32. The method of claim 31, wherein the plant toxin is gelonin.

33. The method of claim 20, further comprising:
iv) isolating the expressed protein from the plastid.

34. A plastid containing a nucleic acid expression construct of claim 1.

35. A microalgae, macroalgae or progeny thereof, containing the plastid of claim 34.

36. The algae of claim 35, wherein the algae is Chlamydomonas reinhardtii.

37. An isolated fusion protein produced using the method of claim 20.

38. A method of killing a eukaryotic cell comprising contacting the eukaryotic cell with a fusion protein isolated from a plant cell or algae cell of claim 16.

39. A method of killing a eukaryotic cell comprising contacting the eukaryotic cell with a fusion protein isolated from a plant cell or algae cell plastid of claim 17.

40. A method of specifically inhibiting B-cell proliferation comprising treating animal or human cells with a therapeutically effective dose of the fusion protein of claim 37.


SEQUENCE LISTING (14 sequences)

SEQ ID NO: 1 - protein, 289 aa, Artificial Sequence
  Synthetic construct
MSIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPLTFGCGTKLEIKRGGGGSGGGGSGGGGSGGGGSQVQLQQSGPELIKPGASVKMSCKASGYTFTSYVMHWVKQKPGQCLEWIGYINPYNDGTKYNEKFKGKATLTSDKSSSTAYMELSSLTSEDSAVYYCARGTYYYGSRVFDYWGQGTTLTVTVSSASGAGTGTCYDYKDHDGKDHDIDYKDDDDKSR

SEQ ID NO: 2 - DNA, 1182 bp, Pseudomonas aeruginosa
  CDS (4)..(1173)
  CDS (1177)..(1182)
catatggcagaaggtggtagcctagcagctctaactgctcaccaagcttgtcacctaccgctagaaactttcactcgtcatcgccaaccgcgcggttgggaacaactagaacaatgtggttatccggtacaacgtctagttgcactttacctagctgctcgtctatcttggaaccaagttgaccaagtaatccgcaacgcactagcaagccctggtagcggtggtgacctaggtgaagctatccgcgaacaaccggaacaagcacgtctagcactaactctagcagcagcagaaagcgaacgcttcgttcgtcaaggtactggtaacgacgaagcaggtgctgcaaacgcagacgtagtaagcctaacttgtccggttgcagcaggtgaatgtgctggtccggctgacagcggtgacgcactactagaacgcaactatcctactggtgctgaattccttggtgacggtggtgacgttagcttcagcactcgcggtacgcaaaactggaccgtggaacgcctacttcaagctcaccgccaactagaagaacgcggttatgtattcgttggttaccacggtactttccttgaagctgctcaaagcatcgttttcggtggtgtacgcgctcgcagccaagaccttgacgctatctggcgcggtttctatatcgcaggtgatccggctctagcatacggttacgcacaagaccaagaacctgacgcacgcggtcgtatccgcaacggtgcactactacgtgtttatgtaccgcgctctagcctaccgggtttctaccgcactagcctaactctagcagctccggaagctgctggtgaagttgaacgtctaatcggtcatccgctaccgctacgcctagacgcaatcactggtcctgaagaagaaggtggtcgcctagaaactattcttggttggccgctagcagaacgcactgtagtaattccttctgctatccctactgacccgcgcaacgttggtggtgaccttgacccgtcaagcatccctgacaaggaacaagctatcagcgcactaccggactacgcaagccaacctggtaaaccgccgcgcgaagacctaaagggtacctgttacgattataaagatcacgatggtgattacaaagatcacgatattgattataaagatgatgatgataaataatctaga

SEQ ID NO: 3 - protein, 392 aa, Pseudomonas aeruginosa
MAEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLKGTCYDYKDHDGDYKDHDIDYKDDDDKSR

SEQ ID NO: 4 - DNA, 2022 bp, Artificial Sequence
  Synthetic construct
catatgatagttatgacacaagctgcaccatctattcctgttactcctggagaatcagtatcaatctcatgtcgttctagtaaaagtcttctgaatagtaatggtaacacttacttatattggttcctgcaacgtccaggccaatctcctcaacttctgatttatcgtatgtcaaaccttgcttcaggtgttccagaccgtttcagtggtagtggttcaggaactgctttcacactgagaatcagtagagtagaagctgaagatgtaggtgtttattactgtatgcaacatttagaatatcctcttactttcggttgtggtacaaaactggaaatcaaacgaggaggaggaggatctggaggaggaggatctggaggaggcggatcaggaggaggtggttcacaagttcaacttcaacaatctggacctgaactgattaaacctggtgcttcagtaaaaatgtcatgtaaagcttctggatacacattcactagctatgttatgcactgggtaaaacaaaaacctggtcaatgtcttgaatggattggatatattaatccttacaatgatggtactaaatacaatgaaaaattcaaaggtaaagctacactgacttcagacaaatcatcaagcacagcttacatggaacttagcagcctgacatctgaagactctgcagtttattactgtgcaagaggtacttattactacggtagtcgtgtatttgactactggggccaaggtacaactcttacagttacagtttcatctgcttctggtgctggtaccagttctggtggcggtggcagtagtggtggtggcggtagtagtggtggcggtggcatggcagaaggtggtagcctagcagctctaactgctcaccaagcttgtcacctaccgctagaaactttcactcgtcatcgccaaccgcgcggttgggaacaactagaacaatgtggttatccggtacaacgtctagttgcactttacctagctgctcgtctatcttggaaccaagttgaccaagtaatccgcaacgcactagcaagccctggtagcggtggtgacctaggtgaagctatccgcgaacaaccggaacaagcacgtctagcactaactctagcagcagcagaaagcgaacgcttcgttcgtcaaggtactggtaacgacgaagcaggtgctgcaaacgcagacgtagtaagcctaacttgtccggttgcagcaggtgaatgtgctggtccggctgacagcggtgacgcactactagaacgcaactatcctactggtgctgaattccttggtgacggtggtgacgttagcttcagcactcgcggtacgcaaaactggaccgtggaacgcctacttcaagctcaccgccaactagaagaacgcggttatgtattcgttggttaccacggtactttccttgaagctgctcaaagcatcgttttcggtggtgtacgcgctcgcagccaagaccttgacgctatctggcgcggtttctatatcgcaggtgatccggctctagcatacggttacgcacaagaccaagaacctgacgcacgcggtcgtatccgcaacggtgcactactacgtgtttatgtaccgcgctctagcctaccgggtttctaccgcactagcctaactctagcagctccggaagctgctggtgaagttgaacgtctaatcggtcatccgctaccgctacgcctagacgcaatcactggtcctgaagaagaaggtggtcgcctagaaactattcttggttggccgctagcagaacgcactgtagtaattccttctgctatccctactgacccgcgcaacgttggtggtgaccttgacccgtcaagcatccctgacaaggaacaagctatcagcgcactaccggactacgcaagccaacctggtaaaccgccgcgcgaagacctaaagggtacatgttacgattataaagatcacgatggtgattacaaagatcacgatattgattataaagatgatgatgataaataatctaga

SEQ ID NO: 5 - protein, 670 aa, Artificial Sequence
  Synthetic construct
MIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPLTFGCGTKLEIKRGGGGSGGGGSGGGGSGGGGSQVQLQQSGPELIKPGASVKMSCKASGYTFTSYVMHWVKQKPGQCLEWIGYINPYNDGTKYNEKFKGKATLTSDKSSSTAYMELSSLTSEDSAVYYCARGTYYYGSRVFDYWGQGTTLTVTVSSASGAGTSSGGGGSSGGGGSSGGGGMAEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLKGTCYDYKDHDGDYKDHDIDYKDDDDK

SEQ ID NO: 6 - DNA, 1215 bp, Artificial Sequence
  Synthetic construct
catatgtggggtacattccttaaagaagctggtcaaggtgctaaagacatgtggagagcttaccaagacatgaaagaagctaactaccgtggtgcagacaaatacttccacgctcgtggtaactatgacgctgctcgacgtggtcctggtggtgcttgggctgctaaagtaatcagtaacgctagagaaactattcaaggtatcacagaccctctttttaaaggtatgacacgtgaccaagtacgtgaagattctaaagctgaccaatttgctaacgaatggggtcgtagcggtaaagaccctaaccacttcagacctgctggtcttcctgacaaatactcaggtggtggtggttcaccatgggaaaatttatattttcaatcaggtttagatacagtttcattttcaacaaaaggtgctacatatattacatacgttaactttttaaatgaattacgtgttaaattaaaaccagaaggtaattcacatggtattccattattacgtaaaaaatgtgatgatccaggtaaatgttttgttttagttgctttatcaaatgataatggtcaattagctgaaattgctattgatgttacatcagtttatgttgttggttatcaagttcgtaatcgttcatatttttttaaagatgctccagatgctgcttatgaaggtttatttaaaaatacaattaaaacacgtttacattttggtggttcatatccatcattagaaggtgaaaaagcttatcgtgaaacaacagatcttggtattgaaccacttcgtatcggcatcaaaaaacttgacgaaaacgcgatcgacaactacaaaccaacagaaatcgcgagctctcttcttgttgtaatccaaatggtaagcgaagcggcacgtttcacattcatcgaaaaccaaattcgtaacaacttccaacaacgtatccgtccagcgaacaacacaatctctcttgaaaacaaatggggcaaacttagcttccaaatccgtacaagcggtgcgaacggtatgttcagcgaagcggtagaacttgaacgcgcgaacggcaaaaaatactacgtaactgcggtagatcaagtaaaaccaaaaatcgcacttcttaaattcgtagacaaagacccagaaggtacctgttacgattataaagatcacgatggtgattacaaagatcacgatattgattataaagatgatgatgataaa

SEQ ID NO: 7 - protein, 405 aa, Artificial Sequence
  Synthetic construct
HMWGTFLKEAGQGAKDMWRAYQDMKEANYRGADKYFHARGNYDAARRGPGGAWAAKVISNARETIQGITDPLFKGMTRDQVREDSKADQFANEWGRSGKDPNHFRPAGLPDKYSGGGGSPWENLYFQSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPEGTCYDYKDHDGDYKDHDIDYKDDDDK

SEQ ID NO: 8 - DNA, 846 bp, Artificial Sequence
  Synthetic construct
catatgggtttagatacagtttcattttcaacaaaaggtgctacatatattacatacgttaactttttaaatgaattacgtgttaaattaaaaccagaaggtaattcacatggtattccattattacgtaaaaaatgtgatgatccaggtaaatgttttgttttagttgctttatcaaatgataatggtcaattagctgaaattgctattgatgttacatcagtttatgttgttggttatcaagttcgtaatcgttcatatttttttaaagatgctccagatgctgcttatgaaggtttatttaaaaatacaattaaaacacgtttacattttggtggttcatatccatcattagaaggtgaaaaagcttatcgtgaaacaacagatcttggtatcgaaccacttcgcatcggcatcaaaaaacttgacgaaaacgcgatcgacaactacaaaccaacagaaatcgcgagctctcttcttgttgtaatccaaatggtaagcgaagcggcacgtttcacattcatcgaaaaccaaattcgtaacaacttccaacaacgtatccgtccagcgaacaacacaatctctcttgaaaacaaatggggcaaacttagcttccaaatccgtacaagcggtgcgaacggtatgttcagcgaagcggtagaacttgaacgcgcgaacggcaaaaaatactacgtaactgcggtagatcaagtaaaaccaaaaatcgcacttcttaaattcgtagacaaagacccagaaggtacctgttacgattataaagatcacgatggtgattacaaagatcacgatattgattataaagatgatgatgataaataatctaga

SEQ ID NO: 9 - protein, 281 aa, Artificial Sequence
  Synthetic construct
HMGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPEGTCYDYKDHDGDYKDHDIDYKDDDDKSR

SEQ ID NO: 10 - DNA, 1668 bp, Artificial Sequence
  Synthetic construct
catatgtcaatagttatgacacaagctgcaccatctattcctgttactcctggagaatcagtatcaatctcatgtcgttctagtaaaagtcttctgaatagtaatggtaacacttacttatattggttcctgcaacgtccaggccaatctcctcaacttctgatttatcgtatgtcaaaccttgcttcaggtgttccagaccgtttcagtggtagtggttcaggaactgctttcacactgagaatcagtagagtagaagctgaagatgtaggtgtttattactgtatgcaacatttagaatatcctcttactttcggttgtggtacaaaactggaaatcaaacgaggaggaggaggatctggaggaggaggatctggaggaggcggatcaggaggaggtggttcacaagttcaacttcaacaatctggacctgaactgattaaacctggtgcttcagtaaaaatgtcatgtaaagcttctggatacacattcactagctatgttatgcactgggtaaaacaaaaacctggtcaatgtcttgaatggattggatatattaatccttacaatgatggtactaaatacaatgaaaaattcaaaggtaaagctacactgacttcagacaaatcatcaagcacagcttacatggaacttagcagcctgacatctgaagactctgcagtttattactgtgcaagaggtacttattactacggtagtcgtgtatttgactactggggccaaggtacaactcttacagttacagtttcatctgcttctggtgctggtacctcttcaggtggtggtggttcaggtggtggtggttctggtttagatacagtttcattttcaacaaaaggtgctacatatattacatacgttaactttttaaatgaattacgtgttaaattaaaaccagaaggtaattcacatggtattccattattacgtaaaaaatgtgatgatccaggtaaatgttttgttttagttgctttatcaaatgataatggtcaattagctgaaattgctattgatgttacatcagtttatgttgttggttatcaagttcgtaatcgttcatatttttttaaagatgctccagatgctgcttatgaaggtttatttaaaaatacaattaaaacacgtttacattttggtggttcatatccatcattagaaggtgaaaaagcttatcgtgaaacaacagatcttggtatcgaaccacttcgcatcggcatcaaaaaacttgacgaaaacgcgatcgacaactacaaaccaacagaaatcgcgagctctcttcttgttgtaatccaaatggtaagcgaagcggcacgtttcacattcatcgaaaaccaaattcgtaacaacttccaacaacgtatccgtccagcgaacaacacaatctctcttgaaaacaaatggggcaaacttagcttccaaatccgtacaagcggtgcgaacggtatgttcagcgaagcggtagaacttgaacgcgcgaacggcaaaaaatactacgtaactgcggtagatcaagtaaaaccaaaaatcgcacttcttaaattcgtagacaaagacccagaaggtacctgttacgattataaagatcacgatggtgattacaaagatcacgatattgattataaagatgatgatgataaataatctaga

SEQ ID NO: 11 - protein, 554 aa, Artificial Sequence
  Synthetic construct
MSIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPLTFGCGTKLEIKRGGGGSGGGGSGGGGSGGGGSQVQLQQSGPELIKPGASVKMSCKASGYTFTSYVMHWVKQKPGQCLEWIGYINPYNDGTKYNEKFKGKATLTSDKSSSTAYMELSSLTSEDSAVYYCARGTYYYGSRVFDYWGQGTTLTVTVSSASGAGTSSGGGGSGGGGSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPEGTCYDYKDHDGDYKDHDIDYKDDDDKSR

SEQ ID NO: 12 - DNA, 2400 bp, Artificial Sequence
  Synthetic construct
catatgatagttatgacacaagctgcaccatctattcctgttactcctggagaatcagtatcaatctcatgtcgttctagtaaaagtcttctgaatagtaatggtaacacttacttatattggttcctgcaacgtccaggccaatctcctcaacttctgatttatcgtatgtcaaaccttgcttcaggtgttccagaccgtttcagtggtagtggttcaggaactgctttcacactgagaatcagtagagtagaagctgaagatgtaggtgtttattactgtatgcaacatttagaatatcctcttactttcggttgtggtacaaaactggaaatcaaacgaggaggaggaggatctggaggaggaggatctggaggaggcggatcaggaggaggtggttcacaagttcaacttcaacaatctggacctgaactgattaaacctggtgcttcagtaaaaatgtcatgtaaagcttctggatacacattcactagctatgttatgcactgggtaaaacaaaaacctggtcaatgtcttgaatggattggatatattaatccttacaatgatggtactaaatacaatgaaaaattcaaaggtaaagctacactgacttcagacaaatcatcaagcacagcttacatggaacttagcagcctgacatctgaagactctgcagtttattactgtgcaagaggtacttattactacggtagtcgtgtatttgactactggggccaaggtacaactcttacagttacagtttcatctgcttctggtgctagatctccaaaatcttgtgacaaaactcacacatgtccaccttgtccagcacctgaactacttggtggtccttcagttttcctattcccaccaaaaccaaaagacacactaatgatctcacgtacacctgaagttacatgtgtagtagtagacgtaagtcacgaagaccctgaagttaaattcaactggtacgtagacggtgtagaagtacataatgcaaaaactaaacctcgtgaagaacaatacaacagtacttaccgtgtagttagtgttctaacagttcttcaccaagactggcttaatggtaaagaatacaaatgtaaagtttcaaacaaagcactaccagcaccaatcgaaaaaacaatctcacaattgggtaccagttctggtggcggtggcagtagtggtggtggcggtagtagtggtggcggtggcatggcagaaggtggtagcctagcagctctaactgctcaccaagcttgtcacctaccgctagaaactttcactcgtcatcgccaaccgcgcggttgggaacaactagaacaatgtggttatccggtacaacgtctagttgcactttacctagctgctcgtctatcttggaaccaagttgaccaagtaatccgcaacgcactagcaagccctggtagcggtggtgacctaggtgaagctatccgcgaacaaccggaacaagcacgtctagcactaactctagcagcagcagaaagcgaacgcttcgttcgtcaaggtactggtaacgacgaagcaggtgctgcaaacgcagacgtagtaagcctaacttgtccggttgcagcaggtgaatgtgctggtccggctgacagcggtgacgcactactagaacgcaactatcctactggtgctgaattccttggtgacggtggtgacgttagcttcagcactcgcggtacgcaaaactggaccgtggaacgcctacttcaagctcaccgccaactagaagaacgcggttatgtattcgttggttaccacggtactttccttgaagctgctcaaagcatcgttttcggtggtgtacgcgctcgcagccaagaccttgacgctatctggcgcggtttctatatcgcaggtgatccggctctagcatacggttacgcacaagaccaagaacctgacgcacgcggtcgtatccgcaacggtgcactactacgtgtttatgtaccgcgctctagcctaccgggtttctaccgcactagcctaactctagcagctccggaagctgctggtgaagttgaacgtctaatcggtcatccgctaccgctacgcctagacgcaatcactggtcctgaagaagaaggtggtcgcctagaaactattcttggttggccgctagcagaacgcactgtagtaattccttctgctatccctactgacccgcgcaacgttggtggtgaccttgacccgtcaagcatccctgacaaggaacaagctatcagcgcactaccggactacgcaagccaacctggtaaaccgccgcgcgaagacctaaagggtaccggtgaaaacttatactttcaaggttcaggtggtggtggatctgattataaagatgatgatgacaaaggaaccggttaatctaga

SEQ ID NO: 13 - protein, 799 aa, Artificial Sequence
  Synthetic construct
HMIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPLTFGCGTKLEIKRGGGGSGGGGSGGGGSGGGGSQVQLQQSGPELIKPGASVKMSCKASGYTFTSYVMHWVKQKPGQCLEWIGYINPYNDGTKYNEKFKGKATLTSDKSSSTAYMELSSLTSEDSAVYYCARGTYYYGSRVFDYWGQGTTLTVTVSSASGARSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISQLGTSSGGGGSSGGGGSSGGGGMAEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLKGTGENLYFQGSGGGGSDYKDDDDKGTGSR

SEQ ID NO: 14 - protein, 6 aa, Artificial Sequence
  Synthetic construct
HHHHHH